(19) United States
(12) Patent Application Publication
(10) Pub. No.: US 2026/0279580 A1
(43) Pub. Date: Sep. 17, 2026

(54) SYSTEMS FOR PREDICTING CARDIAC DISEASE STATUS AND ECHOCARDIOGRAPHIC MEASUREMENTS FROM VETERINARY RADIOGRAPHS

(71) Applicants: Tomas Alejandro Reyes, Katy, TX (US); Tabitha Jean Baibos-Reyes, Katy, TX (US)

(72) Inventors: Tomas Alejandro Reyes, Katy, TX (US); Tabitha Jean Baibos-Reyes, Katy, TX (US)

(21) Appl. No.: 19/680,712

(22) Filed: May 18, 2026

Related U.S. Application Data
(63) Continuation-in-part of application No. 18/196,355, filed on May 11, 2023.
(60) Provisional application No. 63/341,102, filed on May 12, 2022.

(51) Int. Cl. G16H 50/20; G16H 30/40

(52) U.S. Cl. CPC G16H 50/20; G16H 30/40

(57) ABSTRACT

Herein disclosed is a computer-implemented method for cardiac assessment in veterinary patients. The computer-implemented method includes receiving a radiograph image corresponding to an animal, preprocessing the radiograph image to generate a preprocessed radiograph image, generating a left atrial enlargement prediction, and providing access to the left atrial enlargement prediction. The left atrial enlargement prediction is generated based on the preprocessed radiograph image using a left atrial enlargement prediction machine learning model. In certain embodiments, the left atrial enlargement prediction includes one or more of a predicted left atrial-to-aortic ratio (LA:Ao) value, a binary B2 classification indicating whether the animal meets criteria for ACVIM Stage B2 classification, or an ACVIM stage classification indicating one or more of Stage B1, Stage B2, Stage C, or Stage D.

### CROSS-REFERENCE TO RELATED APPLICATIONS

This application is a continuation-in-part of, and claims the benefit of U.S. application Ser. No. 18/196,355, titled “SYSTEM OF USING ARTIFICIAL INTELLIGENCE IN CANINE RADIOGRAPHY” filed on May 11, 2023, which claims priority to U.S. Provisional Application No. 63/341,102, titled “SYSTEM OF USING ARTIFICIAL INTELLIGENCE IN CANINE RADIOGRAPHY” filed on May 12, 2022, and this application incorporates the entire contents of the above-referenced applications herein by reference.

### TECHNICAL FIELD

The present disclosure relates to artificial intelligence systems and methods for analyzing veterinary radiographic images, and more particularly to systems and methods employing machine learning algorithms to predict cardiac measurements, disease status, and echocardiographic parameters from thoracic radiographs of animals, including felines.

### BACKGROUND

Cardiac disease is a common cause of morbidity and mortality in companion animals, including dogs and cats. Veterinary practitioners often rely on thoracic radiography as a first-line diagnostic tool for evaluating cardiac size and detecting signs of cardiac enlargement. Radiographic measurements such as vertebral heart score (VHS) and vertebral left atrial score (VLAS) provide standardized methods for assessing cardiac dimensions relative to vertebral body length, offering objective metrics that can be compared across patients and over time.

While radiographic scoring systems have been validated for use in dogs, their application to feline patients presents additional challenges. Feline cardiac disease, particularly hypertrophic cardiomyopathy, may not produce the same patterns of cardiac silhouette enlargement seen in canine patients. Concentric hypertrophy of the left ventricular wall, which is characteristic of hypertrophic cardiomyopathy in cats, may not substantially alter the overall cardiac silhouette dimensions in early disease stages. As a result, radiographic measurements alone may have limited sensitivity for detecting certain feline cardiac conditions.

Echocardiography remains the gold standard for definitive cardiac assessment in veterinary medicine. Echocardiographic measurements such as the left atrial-to-aortic ratio (LA:Ao) provide direct visualization and quantification of cardiac chamber dimensions. Clinical staging systems, such as those developed for myxomatous mitral valve disease in dogs, may incorporate echocardiographic parameters alongside radiographic findings to classify disease severity and guide treatment decisions. However, echocardiography typically requires specialized equipment and expertise, which may limit its availability in general practice settings.

The application of artificial intelligence and machine learning to medical image analysis has expanded in recent years. Deep learning architectures, including convolutional neural networks and vision transformers, have demonstrated capabilities in extracting features from radiographic images and performing tasks such as landmark detection, segmentation, and classification. These approaches may be trained on datasets containing expert-annotated images to learn patterns associated with anatomical structures or disease states.

Cross-modality prediction, in which a model trained on one imaging modality is used to predict measurements or findings typically obtained from a different modality, represents an area of ongoing research. Such approaches may have the potential to provide preliminary assessments or screening capabilities in situations where the target imaging modality is not readily available.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

One embodiment of the present disclosure includes a computer-implemented method for cardiac assessment in veterinary patients. The computer-implemented method includes receiving a radiograph image corresponding to an animal, preprocessing the radiograph image to generate a preprocessed radiograph image, generating a left atrial enlargement prediction, and providing access to the left atrial enlargement prediction. The left atrial enlargement prediction is generated based on the preprocessed radiograph image using a left atrial enlargement prediction machine learning model.

In certain embodiments of the computer-implemented method, the left atrial enlargement prediction may include one or more of a predicted left atrial-to-aortic ratio (LA:Ao) value, a binary B2 classification indicating whether the animal meets criteria for American College of Veterinary Internal Medicine (ACVIM) Stage B2 classification, or an ACVIM stage classification indicating one or more of Stage B1, Stage B2, Stage C, or Stage D.

In certain embodiments of the computer-implemented method, the computer-implemented method may further include generating a risk-of-progression score indicating a predicted probability of the animal advancing to a worse ACVIM stage within a future time window.

In certain embodiments of the computer-implemented method, preprocessing the radiograph image may include one or more of skew detection, cropping to a region of interest, contrast-limited adaptive histogram equalization (CLAHE), perspective correction, rotation correction, or letterboxed resizing, and the animal may be a canine or a feline.

In certain embodiments of the computer-implemented method, receiving the radiograph image may include receiving a photograph of a radiograph captured by a camera of a mobile device.

In certain embodiments of the computer-implemented method, the computer-implemented method may further include generating a spatial localization overlay indicating one or more regions of the radiograph image that contributed to the left atrial enlargement prediction and displaying the spatial localization overlay on the radiograph image.

In certain embodiments of the computer-implemented method, the computer-implemented method may further include generating a confidence value associated with the left atrial enlargement prediction and providing access to the confidence value.

A second embodiment of the present disclosure includes a system for cardiac assessment in veterinary patients. The system includes at least one processor and memory storing instructions executable by the at least one processor for causing the system to perform operations. The operations include the computer-implemented method. In certain embodiments, one or more of the above-discussed embodiments may be implemented by the system.

A third embodiment of the present disclosure includes a non-transitory computer-readable medium having stored thereon program instructions that are executable by at least one processor for causing the at least one processor to perform operations. The operations include the computer-implemented method. In certain embodiments, one or more of the above-discussed embodiments may be implemented by the non-transitory computer-readable medium.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

The novel features believed characteristic of the embodiments of the present application are outlined in the appended claims. However, the embodiments themselves, as well as a preferred mode of use, and further objectives and advantages thereof, will best be understood by reference to the following detailed description when read in conjunction with the accompanying drawings, wherein:

FIG. 1 shows a system of using artificial intelligence for vertebral heart scale (VHS) and vertebral left atrial score (VLAS) prediction in canine radiography, according to certain embodiments of the present disclosure;

FIG. 2 shows a perspective view of a system displaying prediction lines on an X-ray scan, according to certain embodiments of the present disclosure;

FIG. 3 shows the method steps taken by the system of FIG. 2, according to certain embodiments of the present disclosure;

FIG. 4 shows the overall process operations of the system of FIG. 2, according to certain embodiments of the present disclosure;

FIG. 5 shows a flow diagram of the CNN, according to certain embodiments of the present disclosure;

FIG. 6 shows a flow diagram of the bounding box, according to certain embodiments of the present disclosure;

FIG. 7 shows a flow diagram of the rewards process, according to certain embodiments of the present disclosure;

FIG. 8 shows a flow diagram of the predictive process, according to certain embodiments of the present disclosure;

FIG. 9 shows a flow diagram of the tagging process, according to certain embodiments of the present disclosure;

FIG. 10 shows a flow diagram of the rewards looping process, according to certain embodiments of the present disclosure;

FIG. 11 shows a flow diagram of the rewards calculation process, according to certain embodiments of the present disclosure;

FIG. 12 shows a flow diagram of the AI tagging process, according to certain embodiments of the present disclosure;

FIG. 13 shows a flow diagram of the image tolerance test process, according to certain embodiments of the present disclosure;

FIG. 14 shows a flow diagram of the image check process, according to certain embodiments of the present disclosure;

FIG. 15 shows a flow diagram of the tolerance test within the bounding boxes process, according to certain embodiments of the present disclosure;

FIG. 16 shows a flow diagram of the AI prediction confidence process, according to certain embodiments of the present disclosure;

FIG. 17 shows a block diagram of a computing system, according to certain embodiments of the present disclosure;

FIG. 18 shows a flow diagram of a preprocessing process for radiograph images, according to certain embodiments of the present disclosure;

FIG. 19 shows a flow diagram of a veterinary cardiac assessment process, according to certain embodiments of the present disclosure;

FIG. 20 shows a flow diagram of a veterinary disease prediction process, according to certain embodiments of the present disclosure;

FIG. 21 shows a block diagram of a two-stage prediction system for LA:Ao and B2 prediction, according to certain embodiments of the present disclosure;

FIG. 22 shows a flow diagram of an explainability process for cross-modality prediction, according to certain embodiments of the present disclosure; and

FIG. 23 shows a block diagram of a training system for the AI models, according to certain embodiments of the present disclosure.

While the systems and methods of use of the present application are susceptible to various modifications and alternative forms, specific embodiments thereof have been shown by way of example in the drawings and are herein described in detail. It should be understood, however, that the description herein of specific embodiments is not intended to limit the invention to the particular embodiments disclosed, but on the contrary, the intention is to cover all modifications, equivalents, and alternatives falling within the spirit and scope of the present application as defined by the appended claims.

### DETAILED DESCRIPTION

Illustrative embodiments of the systems and methods of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The systems and methods of use will be understood, both as to its structure and operation, from the accompanying drawings, taken in conjunction with the accompanying description. Several embodiments are presented herein. It should be understood that various components, parts, and features of the different embodiments may be combined and/or interchanged with one another, all of which are within the scope of the present application, even though not all variations and particular embodiments are shown in the drawings. It should also be understood that the mixing and matching of features, elements, and/or functions between various embodiments are expressly contemplated herein so that one of ordinary skill in the art would appreciate from this disclosure that the features, elements, and/or functions of one embodiment may be incorporated into another embodiment as appropriate unless described otherwise.

As used herein, the term “coupled” should be understood to include any direct or indirect connection between two things, such as a wired, mechanical, or other physical connection, a wireless or other non-physical connection, a connection allowing for fluid communication, a connection enabling a transfer of energy, a connection enabling the transfer of information, or any combination thereof. Furthermore, the words “comprising” and any form of comprising, such as “comprise” and “comprises,” “having” and any form of having, such as “has” and “have,” “including” and any form of including, such as “includes” and “include,” or “containing” and any form of containing, such as “contains” and “contain,” are inclusive and open-ended and do not exclude additional, unrecited elements or method steps.

As used herein, the term “at least one of” is synonymous with “one or more of.” For example, the phrase “at least one of A, B, and C” means any one of A, B, and C, or any combination of any two or more of A, B, and C. For example, “at least one of A, B, and C” includes one or more of A alone; one or more of B alone; one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of.” For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F. The phrases “at least one of A, B, and C,” “at least one of A, B, or C,” “one or more of A, B, and C,” and “one or more of A, B, or C” have the same meaning as used herein.

As used herein, the terms “animal” and “animals” may refer to any member of the Animalia kingdom except humans. As used herein, the terms “feline” and “felines” may refer to any member of the Felidae family. As used herein, the terms “canine” and “canines” may refer to any dog-like canid animal in the subfamily Caninae including but not limited to dogs, wolves, coyotes, and jackals.

Artificial intelligence (AI) is intelligence demonstrated by machines, as opposed to the intelligence of humans and other animals. Example tasks in which this is done include speech recognition, computer vision, translation between (natural) languages, as well as other mappings of inputs. Machine learning (ML), a fundamental concept of AI research since the field's inception, is the study of computer algorithms that improve automatically through experience.

Artificial intelligence in healthcare is an overarching term used to describe the use of machine-learning algorithms and software, or artificial intelligence (AI), to mimic human cognition in the analysis, presentation, and comprehension of complex medical and healthcare data. Specifically, AI is the ability of computer algorithms to approximate conclusions based solely on input data. The primary aim of health-related AI applications is to analyze relationships between clinical techniques and patient outcomes. AI programs are applied to practices such as diagnostics, treatment protocol development, drug development, personalized medicine, and patient monitoring and care. What differentiates AI technology from traditional technologies in healthcare is the ability to gather data, process it, and produce a well-defined output for the end-user. AI does this through machine learning algorithms and deep learning. These processes can recognize patterns in behavior and create their logic. To gain useful insights and predictions, machine learning models must be trained using extensive amounts of input data.

Machine learning is a branch of artificial intelligence (AI) and computer science that focuses on the use of data and algorithms to imitate the way that humans learn, gradually improving its accuracy.

Reinforcement learning (RL) is a machine learning training method based on rewarding desired behaviors and/or punishing undesired ones. In general, a reinforcement learning agent can perceive and interpret its environment, take actions and learn through trial and error. Reinforcement learning (RL) is concerned with how intelligent agents ought to take action in an environment to maximize the notion of cumulative reward.

Deep learning is part of artificial intelligence (AI) based on artificial neural networks with representation learning. Learning can be supervised, semi-supervised, or unsupervised. Deep-learning architectures can be applied to medical image analysis.

Within Deep Learning, a Convolutional Neural Network (CNN) is a type of artificial neural network, which is widely used for image/object recognition and classification. Deep Learning thus recognizes objects in an image by using a CNN. CNNs are particularly useful for finding patterns in images to recognize objects, classes, and categories. A CNN is a kind of network architecture for deep learning algorithms and is specifically used for image recognition and tasks that involve the processing of pixel data.

Radiography is a procedure that uses a type of high-energy radiation to take pictures of areas inside the body. Radiation passes through the body onto film or a computer, where the pictures are made. It is used to diagnose or treat patients by recording images of the internal structure of the body to assess the presence or absence of disease, foreign objects, and structural damage or anomaly. During a radiographic procedure, an X-ray beam is passed through the body. Radiography includes but is not limited to X-rays, computed tomography (CT), fluoroscopy, and nuclear medicine including positron emission tomography (PET).

Vertebral heart scale (VHS) is a standardized radiographic measurement used to assess cardiac size in animals by comparing the dimensions of the cardiac silhouette to the length of thoracic vertebrae. VHS may be calculated by measuring the long axis of the heart from the carina to the cardiac apex and the short axis perpendicular to the long axis at its widest point, then comparing these measurements to the length of vertebral bodies starting at the fourth thoracic vertebra (T4). In canines, a normal VHS typically ranges from approximately 8.5 to 10.5 vertebrae, though breed-specific variations exist. Vertebral left atrial score (VLAS) is a complementary measurement that specifically assesses left atrial size by measuring from the carina to the caudal border of the left atrium where it intersects with the dorsal aspect of the caudal vena cava. VLAS values exceeding approximately 2.3 vertebrae may indicate left atrial enlargement. Both VHS and VLAS are clinically significant because they provide objective, reproducible metrics for detecting cardiomegaly and left atrial enlargement, which are indicators of cardiac disease progression and may influence treatment decisions.

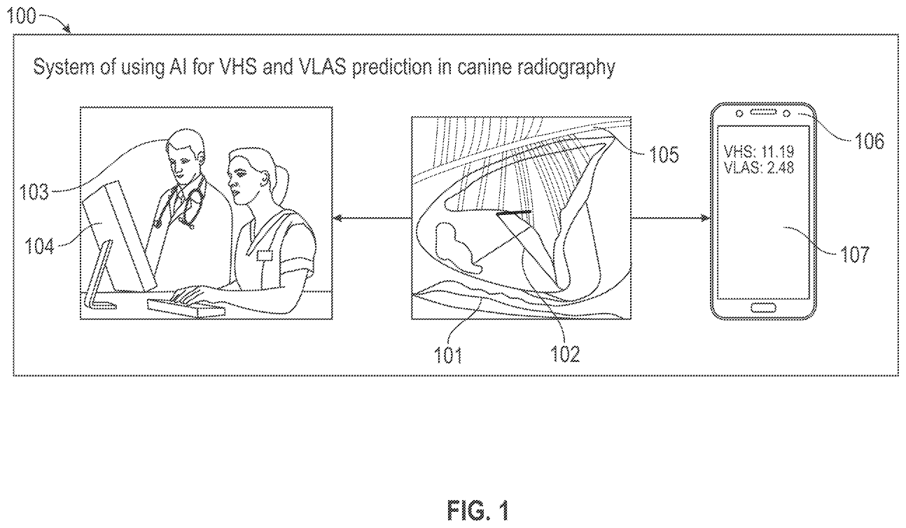

Referring now to the drawings wherein reference characters identify corresponding or similar elements throughout the several views, FIGS. 1-15 depict a system of using convolutional neural networks and reinforcement learning for VHS and VLAS prediction in canine radiography 100. It will be appreciated that said system 100 overcomes one or more of the above-listed problems commonly associated with conventional radiography. In addition, it should be appreciated that more or fewer of such components may be included in different embodiments of the system 100.

The system of using AI for VHS and VLAS prediction in canine radiography 100 comprises a canine 101 needing vertebral heart scale (VHS) and vertebral left atrial score (VLAS) prediction in canine radiography 102, which was done by one or more veterinarians 103 using a radiography device 104. The radiography image/s 105 generated by the radiography device 104 are then uploaded onto a digital device 106 running a mobile or online application 107, as shown in FIG. 1.

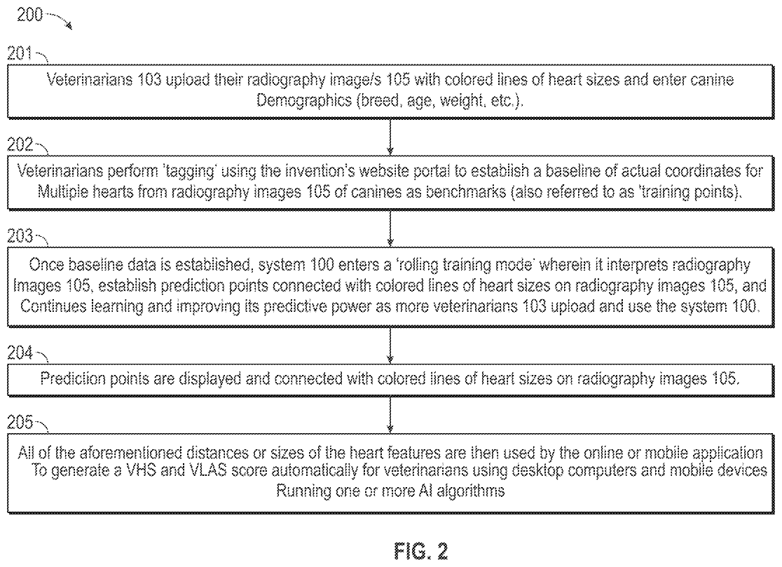

The method of use 200 of the system of using AI for VHS and VLAS prediction in canine radiography 100 for displaying prediction points connected with colored lines of heart sizes on radiography images 105 as shown in FIG. 2. The distance of said colored lines represents distances or sizes of the heart features that can include but are not limited to the apex, ventral border, atrium intersection with caudal vena cava, thoracic vertebrae, and the like. In step 201 veterinarians 103 upload their radiography image/s 105 with colored lines of heart sizes and enter canine demographics (breed, age, weight, etc.). In step 202, veterinarians perform ‘tagging’ using the website portal of the online or mobile application to establish a baseline of actual coordinates for multiple hearts from radiography images 105 of canines as benchmarks (also referred to as ‘training points). In step 203, once baseline data is established, system 100 enters a ‘rolling training mode’ wherein it interprets radiography images 105, establishes prediction points connected with colored lines of heart sizes on radiography images 105, and continues learning and improving its predictive power as more veterinarians 103 upload and use the system 100. In step 204, prediction points are displayed and connected with colored lines of heart sizes on radiography images 105. In step 205, all of the aforementioned distances or sizes of the heart features are then used by the online or mobile application to generate a VHS and VLAS score automatically for veterinarians using desktop computers and mobile devices running one or more AI algorithms.

The AI algorithms include but are not limited to convolutional neural networks (CNN), reinforcement learning (RL), and Deep Learning (DL).

After training, the online or mobile application performs image localization on new x-rays and once an x-ray is uploaded, the AI algorithm/s begin the image localization process on the online or mobile application. The AI algorithm/s first identify specific pixel colors on the image and generates bounding boxes around the points of interest. The image is stored in a grayscale format (a number from 0-255) and by normalizing the image across these values, the program achieves a consistent set of numbers that can allow for pattern comparisons. For example, white inside a normalized X-ray is defined as 1, and a darker area range from 0.0 to 0.99999. CNN performs pattern detection on the images by combining multiple layers with filters ex. edges, shapes, lighting, etc. to detect patterns such as organs and organ locations.

The system 100 may accept radiography images 105 in various digital formats including, but not limited to, DICOM (Digital Imaging and Communications in Medicine), JPEG, PNG, TIFF, and BMP formats. In some embodiments, the system 100 may accept photographs of radiographs captured by a camera on the digital device 106. The radiography images 105 may have a minimum resolution sufficient to identify anatomical landmarks, though higher resolutions may improve prediction accuracy. The system 100 may perform preprocessing operations on uploaded images including normalization, contrast enhancement, noise reduction, rotation correction, and cropping to isolate the region of interest containing the cardiac silhouette and thoracic vertebrae. In cases where an uploaded image does not meet quality thresholds, the system 100 may prompt the user to upload a different image or may provide guidance on improving image quality.

After the CNN process, the RL is performed wherein points are gathered and compared against the benchmarks for accuracy, weighted, and assigned either positive or negative rewards. The RL algorithm interprets grey scale colors on an X-ray and then draws a multitude of lines to define heart measurements. After these lines are applied to the x-ray a ‘shaping function’ draws a radius around each point to represent the distance to its corresponding benchmark. As the distance (measured in pixels) between this radius and a target point increases, the reward decreases and these points are weighted less by the program.

Conversely, as the distance between the radius and a target point decreases, the reward increases, and these points are weighted more and are used to support a final prediction. They are also placed into the program's library for future scans. Using the bounding boxes from image localization, the program establishes sub-images and generates its own tag points, and draws a line between them to establish distances that predict VHS and VLAS scores for the veterinarian automatically.

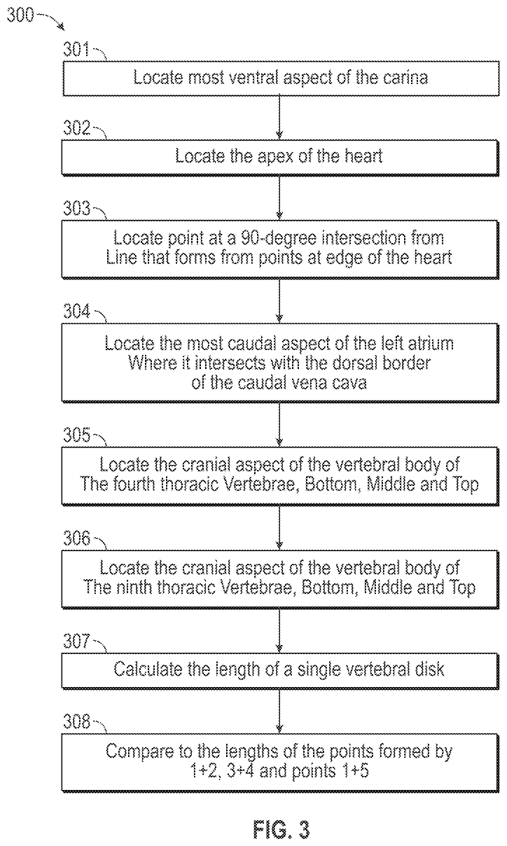

FIG. 3 shows the method steps taken by the AI 300 on digital radiography images uploaded into the system of FIG. 2. This method includes but is not limited to the following steps: in step 301, locating the most ventral aspect of the carina and establishing tags; in step 302, locating the apex of the heart and establishing tags; in step 303, locating a point at a 90-degree intersection from the line that forms from points one and two to the edge of the heart and establishing tags; in step 304, locating the most caudal aspect of the left atrium where it intersects with the dorsal border of the caudal vena cava and establishing tags; in step 305, locating the cranial aspect of the vertebral body of the fourth thoracic vertebrae, bottom, middle, and top and establishing tags; in step 306, locating the cranial aspect of the vertebral body of the ninth thoracic vertebrae, bottom, middle, and top and establishing tags; in step 307, using the assumption that spinal disks 4-9 are evenly spaced, the length of a single vertebral disk is calculated; and in step 308, the calculated vertebral disk length is compared to the lengths of the aforementioned tags and the VHS and VLAS are calculated from the tags.

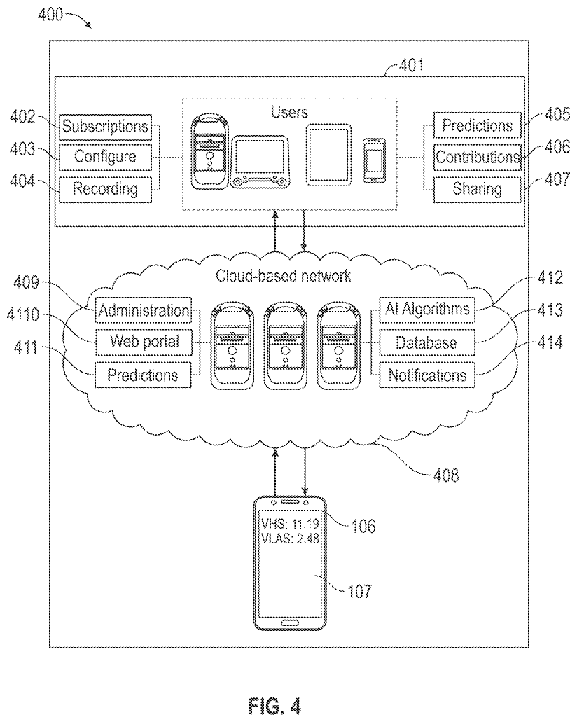

FIG. 4 shows the overall process operations 400 of the system 100. Users 401 have functions that include but are not limited to selecting a subscription type 402; configuring their x-ray imaging software 403 to export compatible digital imagery; having the option to record 404 their benchmark tags for submission; predicting VHS and VLAS 405; contributing radiography images for VHS and VLAS 406; and sharing findings 407 with others via email, SMS, text messages, and the like. A cloud-based network 408 is connected to the users 401 through desktop software or an app and has operations that include but are not limited to administration 409 for administrative routines such as user demographics, payments, and subscription management; a web portal 410 providing an interface that allows image uploading and viewing prediction results; detailed prediction data 411 for viewing statistics for each tag and downloading data; AI algorithms 412 implementing CNN and RL; a historical database 413 providing encrypted storage of imaging and all results; and notifications 414 for stakeholder communications via email, SMS, text messages, and the like. The digital device 106 running a mobile or online application 107 is connected to the cloud-based network 408, which is connected with other users 401.

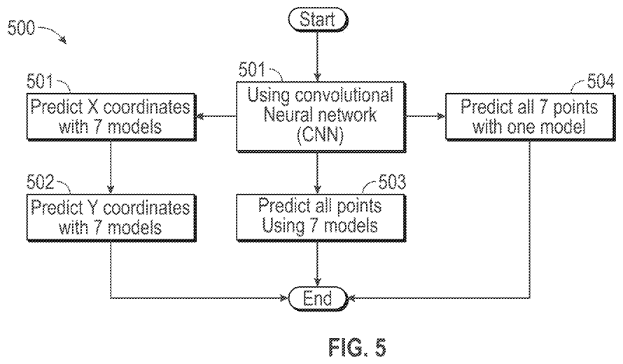

FIG. 5 shows a flow diagram of the method of use of CNN algorithm 500, wherein seven models may be executed to predict accurate tag points. The tag points are in both the X and Y directions. In step 501, a convolutional neural network (CNN) is used. In one path, the CNN predicts X coordinates with 7 models in step 501, followed by predicting Y coordinates with 7 models in step 502. In step 503, all points may be predicted by using 7 models. In step 504, all 7 points may be predicted by 1 model.

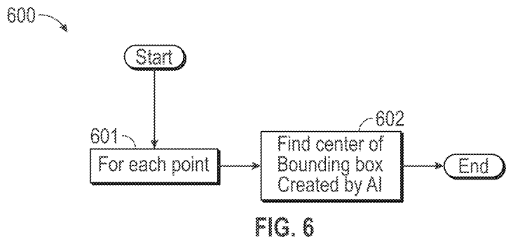

FIG. 6 shows a flow diagram of the bounding box prediction process 600. In step 601, for each tag point, the process iterates. In step 602, the center of the bounding box is generated by the AI.

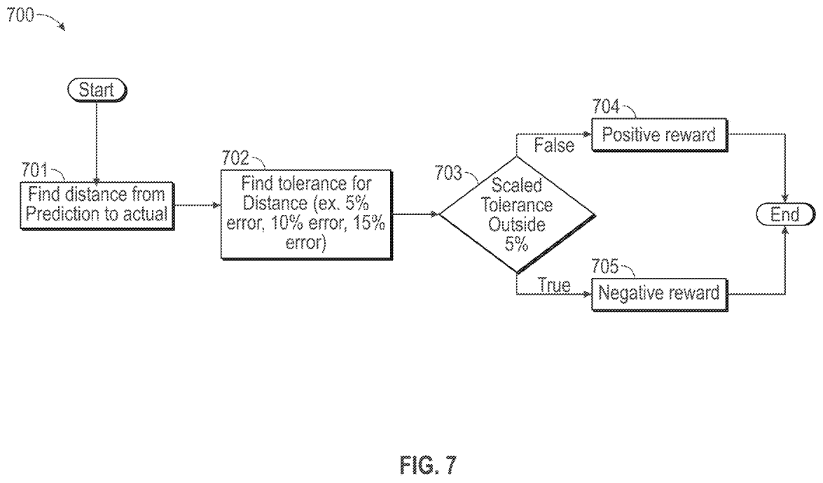

FIG. 7 shows a flow diagram of the rewards process 700. In step 701, the AI determines the distance from an AI-predicted tag to the actual benchmark tag. In step 702, a tolerance for the distance is determined, such as 5% error, 10% error, or 15% error. In step 703, the program determines whether the scaled tolerance is outside 5%. If the scaled tolerance is not outside 5% (False), the program applies a positive reward in step 704. If the scaled tolerance is outside 5% (True), the program applies a negative reward in step 705.

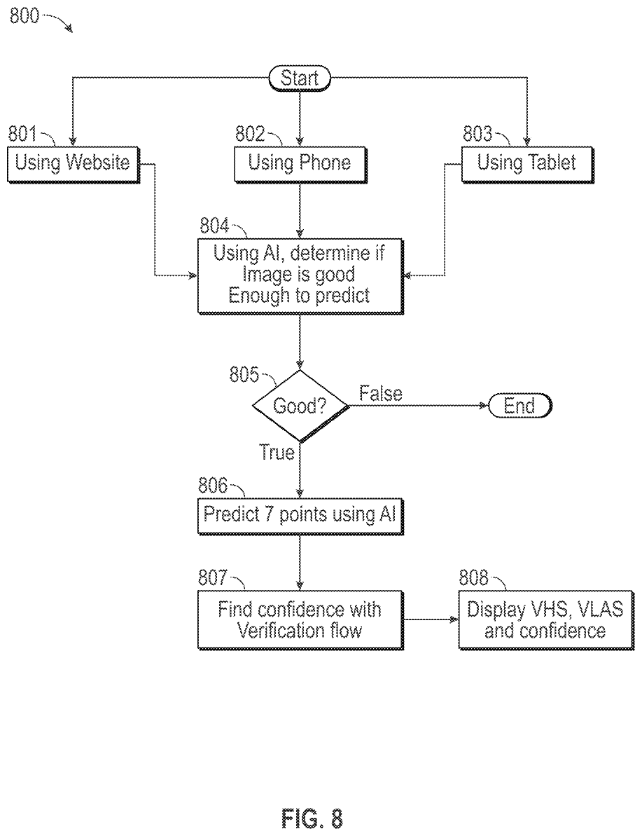

FIG. 8 shows a flow diagram of the predictive process 800 that may be performed on a remote website in step 801, a phone in step 802, or a tablet in step 803. Once an image is uploaded to these platforms, in step 804, AI is used to determine whether the image is good enough for predicting. In step 805, if the image is determined to be good for predicting, the process proceeds; otherwise, the process ends. In step 806, at least seven tag points are predicted using AI. In step 807, a confidence level is found using a verification flow. In step 808, the VHS, VLAS, and confidence level are displayed.

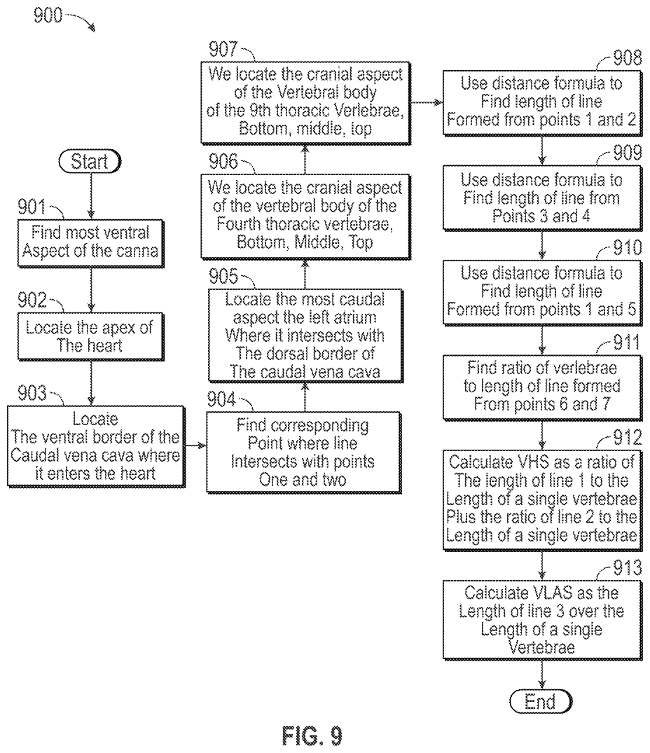

FIG. 9 shows a flow diagram of the tagging process 900 wherein each tag point that is predicted is associated with heart anatomy. For example, the ventral aspect of the carina corresponds to point 1, the apex of the heart corresponds to point 2, and the ventral border of the caudal vena cava corresponds to point 3. The program applies a distance formula between each of the points and calculates the VHS and VLAS as a ratio of specifically associated line lengths that connect these points. In tagging process 900, step 901 includes finding the most ventral aspect of the carina. Step 902 includes locating the apex of the heart. Step 903 includes locating the ventral border of the caudal vena cava where it enters the heart. Step 904 includes finding the corresponding point where the line intersects with points one and two. Step 905 includes locating the most caudal aspect of the left atrium where it intersects with the dorsal border of the caudal vena cava. Step 906 includes locating the cranial aspect of the vertebral body of the fourth thoracic vertebrae, bottom, middle, and top. Step 907 includes locating the cranial aspect of the vertebral body of the ninth thoracic vertebrae, bottom, middle, and top. Step 908 includes using the distance formula to find the length of the line formed from points 1 and 2. Step 909 includes using the distance formula to find the length of the line formed from points 3 and 4. Step 910 includes using the distance formula to find the length of the line formed from points 1 and 5. Step 911 includes finding the ratio of vertebrae to the length of the line formed from points 6 and 7. Step 912 includes VHS calculation as the ratio of the length of line 1 to the length of a single vertebra plus the ratio of line 2 to the length of a single vertebra. Step 913 includes VLAS calculation based on the length of line 3 over the length of a single vertebra.

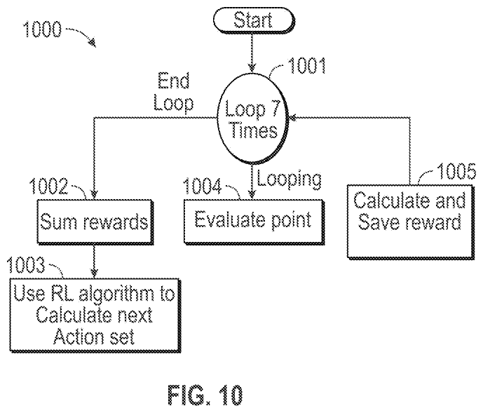

FIG. 10 shows a flow diagram of the rewards looping process 1000 whereby each verified point or tag is assigned a reward. Step 1001 includes a loop 7 times, step 1002 includes sum rewards after ending the loop, step 1003 includes using RL algorithm to calculate the next action set, step 1004 includes evaluating point in each loop of step 1001, followed by step 1005 includes calculating and saving reward before entering into the loop in step 1001.

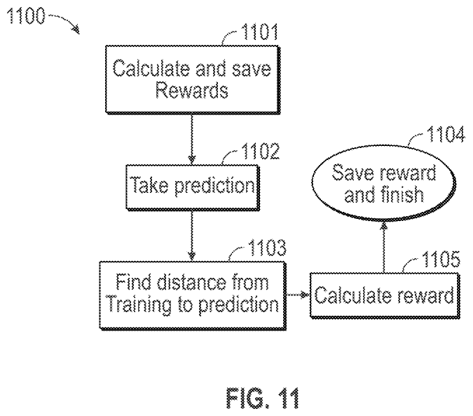

FIG. 11 shows a flow diagram of the rewards calculation process 1100. Step 1101 includes calculating and saving rewards. Step 1102 includes taking a prediction. Step 1103 includes finding the distance from training data to the prediction. Step 1105 includes calculating the reward based on the distance. Step 1104 includes saving the reward and finishing the process.

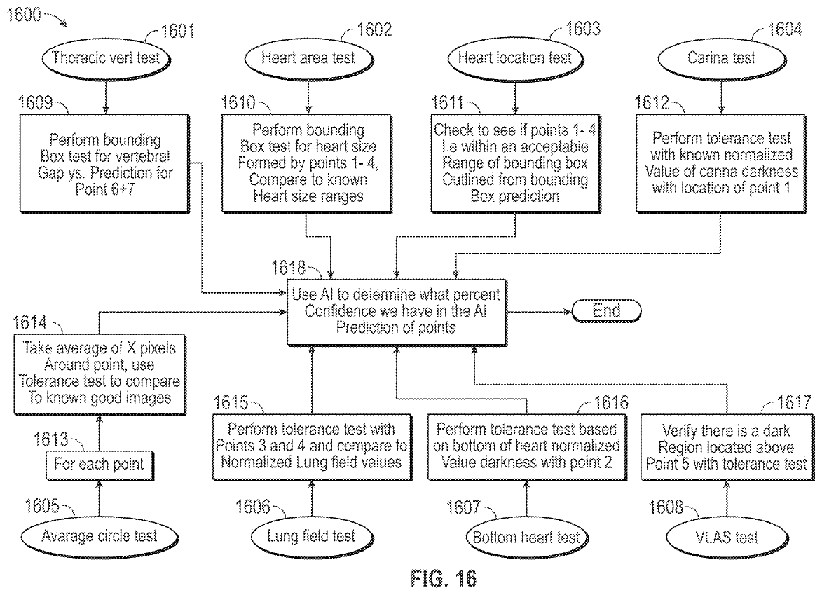

In some embodiments, two or more of the individual confidence tests described in FIG. 16 may be combined to produce a final confidence percentage using various aggregation methods. For example, the results from the thoracic vertebra test 1601, heart area test 1602, heart location test 1603, carina test 1604, average circle test 1605, lung field test 1606, bottom heart test 1607, and VLAS test 1608 may be weighted and summed to produce an overall confidence score. In some embodiments, each test may contribute equally to the final confidence percentage. In other embodiments, certain tests may be weighted more heavily based on their predictive importance or reliability. Weights may be hard-coded or provided via an AI aggregation model. The system may also implement a minimum threshold approach wherein the final confidence is determined by the lowest individual test score, ensuring that a failure in any single test appropriately reduces overall confidence. The aggregated confidence score may be expressed as a percentage from 0% to 100%, with higher values indicating greater reliability of the VHS and VLAS predictions.

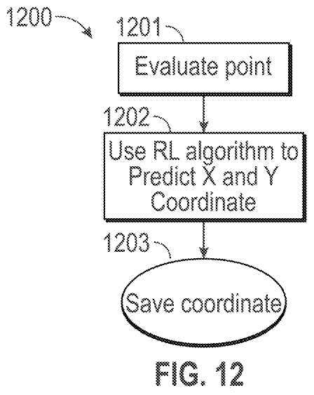

FIG. 12 shows a flow diagram of the AI coordinates tagging process 1200. Step 1201 includes evaluating the point, followed by step 1202 which includes using the RL algorithm to predict X and Y coordinates, followed by step 1203 which includes saving coordinates.

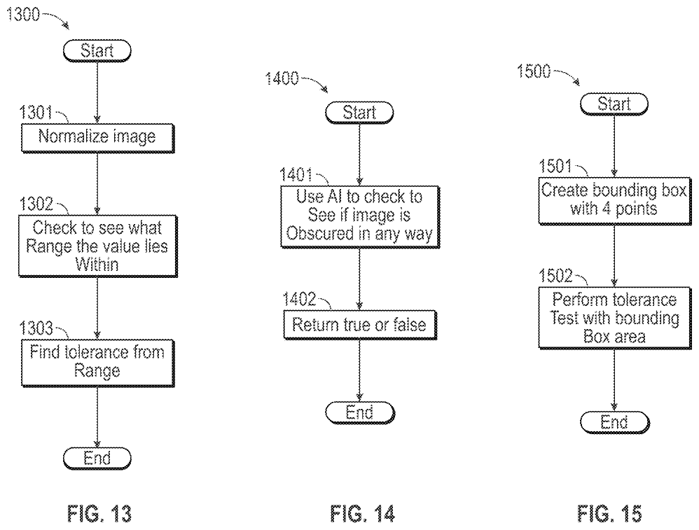

FIG. 13 shows a flow diagram of the image tolerance test process 1300 which involves normalizing an image and determining an acceptable image tolerance range. Step 1301 includes normalizing the image, followed by step 1302 which includes checking to see the range value, followed by step 1303 which includes finding tolerance from the range.

FIG. 14 shows a flow diagram of the image check process 1400 and checking for any potential image obscurities. Step 1401 includes using AI to check for any obscurities in the image, followed by step 1402 which includes returning true or false for the presence of any image obscurities.

FIG. 15 shows a flow diagram of the tolerance test within the bounding boxes process based on four tag points 1500. Step 1501 includes creating a bounding box with 4 points, followed by step 1502 which includes performing a tolerance test with the bounding box area.

FIG. 16 shows a flow diagram of the AI prediction confidence process 1600 wherein every tag point that is recognized as a viable heart feature undergoes normalization, a bounding box test, and a tolerance test that allows the AI to make a confidence determination regarding their validity to predict VHS and VLAS. Using AI to determine what percent of confidence the system has in the AI prediction of points includes the following steps. Step 1601 includes a thoracic vertebra test, which further includes step 1609 of performing a bounding box test for a vertebral gap against prediction for points 6 and 7. Step 1602 includes a heart area test, which further includes step 1610 of performing a bounding box test for heart size formed by points 1 through 4 and comparing to known heart size ranges. Step 1603 includes a heart location test, which further includes step 1611 of performing a bounding box test for heart location formed by points 1 through 4 and checking if they lie within an acceptable range of the bounding box outline. Step 1604 includes a carina test, which further includes step 1612 of performing a tolerance test with a known normalized value of carina darkness with the location of point 1. Step 1605 includes an average circle test, which further includes step 1613 for each point of performing an average circle test, and step 1614 of taking an average of pixels around the point and using a tolerance test to compare to known good images. Step 1606 includes a lung field test, which further includes step 1615 of performing a tolerance test for points 3 and 4 and comparing to normalized lung field values. Step 1607 includes a bottom heart test, which further includes step 1616 of performing a tolerance test based on the bottom of heart normalized value darkness with point 2. Step 1608 includes a VLAS test, which further includes step 1617 of verifying that a dark region is located above point 5 with a tolerance test. All testing pathways converge at step 1618, where AI is used to determine what percent confidence the system has in the AI prediction of points.

In some embodiments, the mobile or online application 107 provides a user interface that displays the predicted VHS value, VLAS value, and confidence score to the veterinarian 103. The user interface may display the radiography image 105 with overlaid prediction points and colored lines indicating the measured distances used to calculate VHS and VLAS. The user interface may also display numerical values for each measurement, reference ranges for normal values, and an indication of whether the predicted values fall within normal ranges or suggest cardiac enlargement. In some embodiments, the user interface may allow the veterinarian 103 to adjust or correct the predicted tag points, and such corrections may be submitted back to the system 100 to improve future predictions through continued learning. The user interface may also provide options to save results, generate reports, share findings with colleagues or specialists, and access historical data for longitudinal tracking of a patient's cardiac measurements over time.

In view of the disclosure provided herein, a mobile application is created by techniques known to those of skill in the art using hardware, languages, and development environments known to the art. Those with skill in the art will recognize that mobile applications are written in several languages including, by way of non-limiting examples, C, C++, CA, Objective-C, Java™, JavaScript, Pascal, Object Pascal, Python™ Ruby, VB.NET, WML, and XHTML/HTML with or without CSS, or combinations thereof. The software is also compatible with a plurality of operating systems such as, but not limited to Windows™ Apple™, and Android™, and compatible with a multitude of hardware platforms such as, but not limited to personal desktops, laptops, tablets, smartphones, and the like. Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for the distribution of mobile applications including, by way of non-limiting examples, Apple® App Store, Google® Play, Chrome web Store, BlackBerry @ App World, App Store for Palm devices, App Catalog for webOS, Windows® Marketplace for Mobile, Ovi Store for Nokia® devices, Samsung® Apps, and Nintendo® DSi Shop.

In some embodiments, a computer program includes a standalone application, which is a program that is run as an independent computer process, not an add-on to an existing process, e.g., not a plug-in. Those with skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. The compilation is often performed, at least in part, to create an executable program. In some embodiments, a computer program includes one or more executable complied applications. In some embodiments, the computer program includes a web browser plugin (e.g., extension, etc.). In computing, a plug-in is one or more software components that add specific functionality to a larger software application. Makers of software applications support plug-ins to enable third-party developers to create abilities that extend an application, to support easily adding no′ features, and to reduce the size of an application. When supported, plug-ins enable customizing the functionality of a software application. For example, plug-ins are commonly used in web browsers to play videos, generate interactivity, scan for viruses, and display particular file types. Those with skill in the art will be familiar with several web browser plug-ins including, Adobes Flashy Player, Microsoft Silverlight®, and Apple® Quick Time®.

In some embodiments, the platforms, systems, media, and methods disclosed herein include software, server, and/or database modules, or use of the same. Given the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

The application of CNNs and RL to AI allows for deep learning of x-ray imaging to take place because the system incorporates a plurality of models and aggregates findings that have been shown to have a higher validity score. These higher validity scores (known as a reward) are then allowed to re-enter the learning knowledge base for deeper learning and continuous improvement of the system.

It is contemplated that certain systems of the present disclosure help veterinarians and technicians to easily capture radiography imaging. Certain embodiments include a mobile application (app) that can be installed on a user's computer (e.g., a phone or tablet) and can use one or more cameras to capture existing scans for inclusion into one or more of the herein-disclosed systems.

It is also contemplated and will be appreciated that certain systems of the present disclosure provides a means to accelerate the radiography process. Utilizing AI as a prescreening method to rule out heart conditions can allow veterinarians to focus on other scans that may be of importance for a dog's survival. This greater efficiency can lead to less wait time for patients and reduced diagnosis and treatment costs.

It should also be appreciated that one of the unique features believed characteristic of the present application is that it facilitates overall improvements in canine VHS and VLAS diagnosis, as [symbol] thousands of veterinarians around the world to contribute to AI learning, hundreds of thousands of canine breeds, types, ages, and sizes can be captured and included. Therefore, veterinarians in one part of the world can benefit from contributions from other parts of the world.

In alternative embodiments, the AI algorithms may include neural network architectures other than CNNs, such as vision transformers, recurrent neural networks, or hybrid architectures combining multiple network types. The system 100 may employ different training approaches including supervised learning, semi-supervised learning, transfer learning from pre-trained models, or self-supervised pretraining on unlabeled radiograph datasets. In some embodiments, the system 100 may be deployed entirely on-device without requiring cloud connectivity, enabling use in low-connectivity environments. In other embodiments, the system 100 may be integrated directly into radiography equipment or Picture Archiving and Communication Systems (PACS). The reinforcement learning component may utilize various reward functions and learning algorithms beyond those specifically described herein. Additionally, while canine cardiac assessment is discussed above by way of example, the underlying architecture and methods may be adapted for use with other species (for example, and without limitation, felines) or for detecting other anatomical features and conditions visible on radiographic images.

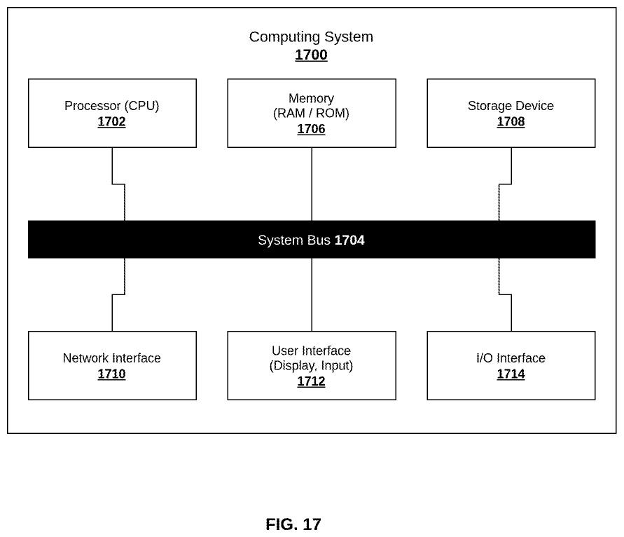

FIG. 17 illustrates a block diagram of a computing system 1700 that may be used to implement one or more of the systems and methods described herein. The computing system 1700 may comprise one or more of a server, a workstation, a desktop computer, a laptop computer, a tablet computer, a smartphone, an embedded computing device, or any other computing device capable of executing the AI algorithms and processing radiographic images as described herein.

The computing system 1700 may include a processor 1702 (also referred to as a central processing unit or CPU). The processor 1702 may comprise any one or more processors, including one or more of a single-core processor, a multi-core processor, a microprocessor, a microcontroller, a digital signal processor (DSP), a field-programmable gate array (FPGA), an application-specific integrated circuit (ASIC), a graphics processing unit (GPU), a tensor processing unit (TPU), a neural processing unit (NPU), or any combination thereof. In some embodiments, the processor 1702 may include multiple processing units operating in parallel or in a distributed configuration. The processor 1702 may execute instructions stored in memory to perform the image processing, machine learning inference, and prediction operations described herein. In certain embodiments, the processor 1702 may include specialized hardware accelerators configured to accelerate neural network computations, matrix operations, or other mathematical operations associated with deep learning inference.

The computing system 1700 may include a memory 1706 coupled to the processor 1702. The memory 1706 may comprise one or more of random access memory (RAM), dynamic random access memory (DRAM), static random access memory (SRAM), synchronous dynamic random access memory (SDRAM), double data rate (DDR) memory, read-only memory (ROM), programmable read-only memory (PROM), erasable programmable read-only memory (EPROM), electrically erasable programmable read-only memory (EEPROM), flash memory, cache memory, register memory, or any combination thereof. The memory 1706 may store executable instructions, operating system code, application code, machine learning model parameters, neural network weights, intermediate computation results, radiographic image data, and other data used during operation of the computing system 1700. In some embodiments, the memory 1706 may include high-bandwidth memory (HBM) or other memory architectures configured to support the data throughput requirements of neural network inference operations.

The computing system 1700 may include a storage device 1708 coupled to the processor 1702. The storage device 1708 may comprise one or more of a hard disk drive (HDD), a solid-state drive (SSD), a hybrid drive, an optical disc drive, a magnetic tape drive, a flash memory device, a memory card, a network-attached storage (NAS) device, a storage area network (SAN) device, or any combination thereof. The storage device 1708 may store any one or more of operating system software, application software, machine learning models, training data, radiographic image archives, prediction results, historical patient data, and other persistent data. In some embodiments, the storage device 1708 may include redundant storage configurations such as RAID (Redundant Array of Independent Disks) for data protection and fault tolerance.

The computing system 1700 may include a network interface 1710 coupled to the processor 1702. The network interface 1710 may comprise one or more of an Ethernet adapter, a Wi-Fi adapter, a cellular modem (including 3G, 4G, LTE, 5G, or other cellular technologies), a Bluetooth adapter, a near-field communication (NFC) interface, a satellite communication interface, or any combination thereof. The network interface 1710 may enable the computing system 1700 to communicate with remote servers, cloud computing platforms, other computing devices, radiography equipment, Picture Archiving and Communication Systems (PACS), veterinary practice management systems, and other networked resources. In some embodiments, the network interface 1710 may support multiple simultaneous network connections and may implement various network protocols including TCP/IP, HTTP, HTTPS, WebSocket, MQTT, HL7, DICOM networking protocols, or other communication protocols.

The computing system 1700 may include a user interface 1712 coupled to the processor 1702. The user interface 1712 may comprise one or more input devices and one or more output devices. Input devices may include one or more of a keyboard, a mouse, a trackpad, a touchscreen, a stylus, a microphone, a camera, a scanner, a barcode reader, a biometric sensor, any other suitable input device, or any combination thereof. Output devices may include one or more of a display (such as an LCD, LED, OLED, or other display technology), a speaker, a haptic feedback device, a printer, any other suitable output device, or any combination thereof. The user interface 1712 may enable veterinarians, technicians, and other users to interact with the computing system 1700, upload radiographic images, view prediction results, adjust settings, and perform other operations. In some embodiments, the user interface 1712 may present graphical user interfaces displaying radiographic images with overlaid prediction points, colored lines indicating measured distances, VHS values, VLAS values, LA:Ao values, confidence scores, and other outputs.

The computing system 1700 may include an input/output (I/O) interface 1714 coupled to the processor 1702. The I/O interface 1714 may comprise one or more of a Universal Serial Bus (USB) port, a Thunderbolt port, a FireWire port, a serial port (such as RS-232 or RS-485), a parallel port, an HDMI port, a DisplayPort, a VGA port, an audio jack, a memory card slot, a general-purpose input/output (GPIO) interface, any other suitable interface modality, or any combination thereof. The I/O interface 1714 may enable the computing system 1700 to connect to external devices including radiography equipment, external storage devices, displays, printers, and other peripheral devices. In some embodiments, the I/O interface 1714 may support direct connection to digital radiography plates, x-ray machines, or other medical imaging equipment for direct image acquisition.

The computing system 1700 may include a system bus 1704 that interconnects the processor 1702, the memory 1706, the storage device 1708, the network interface 1710, the user interface 1712, and the I/O interface 1714. The system bus 1704 may comprise one or more of a data bus, an address bus, a control bus, a peripheral component interconnect (PCI) bus, a PCI Express (PCIe) bus, an Advanced Microcontroller Bus Architecture (AMBA) bus, a HyperTransport bus, a QuickPath Interconnect (QPI), an Infinity Fabric, or any combination thereof. The system bus 1704 may enable data transfer and communication between the various components of the computing system 1700.

In some embodiments, the computing system 1700 may be implemented as a cloud computing platform. Cloud computing implementations may include one or more of Infrastructure as a Service (IaaS), Platform as a Service (PaaS), Software as a Service (SaaS), or Function as a Service (FaaS) deployment models. Cloud computing platforms may include one or more of Amazon Web Services (AWS), Microsoft Azure, Google Cloud Platform (GCP), IBM Cloud, Oracle Cloud, or other cloud service providers. Cloud-based implementations may provide scalable computing resources, load balancing, automatic failover, geographic distribution, and other capabilities. In some embodiments, the computing system 1700 may utilize cloud-based GPU instances or other specialized computing instances configured for machine learning inference.

In some embodiments, the computing system 1700 may be implemented as a distributed computing system comprising multiple computing nodes. Distributed computing implementations may include one or more of a computing cluster, a server farm, a grid computing system, a peer-to-peer network, or a federated computing architecture. Distributed implementations may enable parallel processing of multiple radiographic images, distributed model training, load distribution across multiple geographic locations, and fault-tolerant operation. In some embodiments, the computing system 1700 may implement edge computing architectures wherein initial processing occurs on local devices and additional processing occurs on remote servers.

In some embodiments, the computing system 1700 may implement hybrid computing architectures combining on-device processing with cloud-based processing. Hybrid architectures may enable lightweight preprocessing and initial inference on local devices (such as smartphones or tablets) with more computationally intensive operations performed on cloud servers. Hybrid architectures may provide reduced latency for initial results while enabling access to more powerful computing resources for complex analyses.

The computing system 1700 may execute various artificial intelligence and machine learning algorithms. In some embodiments, the computing system 1700 may execute convolutional neural networks (CNNs) for image feature extraction, landmark detection, and classification tasks. In some embodiments, the computing system 1700 may execute vision transformer architectures for image analysis. In some embodiments, the computing system 1700 may execute reinforcement learning algorithms for prediction refinement and reward-based learning. In some embodiments, the computing system 1700 may execute segmentation networks for cardiac silhouette extraction. The computing system 1700 may utilize machine learning frameworks including one or more of TensorFlow, PyTorch, ONNX Runtime, TensorRT, Core ML, or other inference engines.

In some embodiments, the computing system 1700 may be configured to achieve end-to-end inference latency of less than 20 seconds. End-to-end inference latency may include the time from receipt of a radiographic image through preprocessing, model inference, post-processing, and output generation. Achieving low inference latency may involve one or more of model optimization techniques (such as quantization, pruning, or knowledge distillation), hardware acceleration, efficient data pipelines, caching strategies, or optimized network communication.

In some embodiments, the computing system 1700 may be configured as a clinician-facing tool that outputs measurements and predictions with confidence values. The computing system 1700 may output VHS values, VLAS values, LA:Ao values, disease classifications, ACVIM stage classifications, risk-of-progression scores, and other measurements along with associated confidence intervals or probability values. In some embodiments, the computing system 1700 does not generate treatment recommendations or referral instructions, thereby preserving veterinarian interpretive authority over clinical decision-making. The outputs provided by the computing system 1700 may be presented as objective measurements and predictions that veterinarians may interpret in conjunction with other clinical findings.

In some embodiments, the computing system 1700 may implement security measures including one or more of data encryption (at rest and in transit), user authentication, access control, audit logging, secure boot, trusted execution environments, or compliance with veterinary data protection regulations. In some embodiments, the computing system 1700 may implement backup and disaster recovery capabilities to protect against data loss.

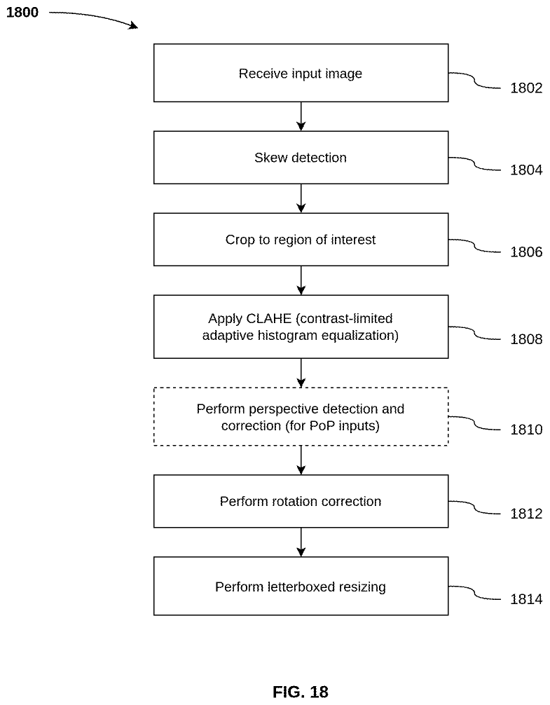

Turning now to FIGS. 18-23, certain embodiments of the depicted systems and methods may extend the above-discussed cardiac assessment capabilities to additional species and additional prediction outputs. The systems may include one or more of a preprocessing pipeline configured to receive radiograph images in various formats including photographs of radiographs and DICOM files, a veterinary cardiac assessment module configured to detect species-specific anatomical landmarks and compute VHS and VLAS measurements using veterinary reference protocols (for example, and without limitation, feline reference protocols), a veterinary disease prediction module configured to predict cardiac disease status from radiographic features that may be independent of conventional scoring systems, a two-stage prediction architecture in which outputs from a VHS and VLAS measurement pipeline are provided as features to a downstream model configured to predict echocardiography-derived metrics such as left atrial-to-aortic ratio (LA:Ao) and clinical staging classifications, an explainability module configured to generate spatial localization overlays indicating regions of the radiograph that contributed to model predictions, and a training system configured to receive paired radiograph and echocardiography studies along with cardiologist annotations for supervised learning. In some embodiments, the systems may be deployed via one or more of cloud-based platforms, on-device inference, embedded integration with radiography equipment, or hybrid edge and cloud architectures.

Existing approaches to cardiac assessment in veterinary patients present several challenges that may limit their clinical utility. Echocardiography, which provides direct visualization and quantification of cardiac chamber dimensions including left atrial-to-aortic ratio (LA:Ao), remains the gold standard for definitive cardiac assessment and staging. However, echocardiography may require specialized equipment and expertise that are not uniformly available across veterinary practice settings. Board-certified veterinary cardiologists are often concentrated in major urban and academic centers, and wait times for non-urgent echocardiography appointments may range from two to six weeks or longer in some regions. The cost of cardiologist-performed echocardiography may present a barrier for some pet owners seeking definitive cardiac staging. These access and cost constraints may result in delayed diagnosis of conditions such as ACVIM Stage B2 myxomatous mitral valve disease, which represents the threshold for treatment with medications that have been shown to delay onset of congestive heart failure and extend survival.

Radiographic scoring systems such as vertebral heart score (VHS) and vertebral left atrial score (VLAS) provide objective metrics that can be obtained from thoracic radiographs without requiring echocardiography. However, published studies have indicated that radiographic scores alone may have limitations for certain applications. In feline patients, VHS may have moderate sensitivity because hypertrophic cardiomyopathy, the dominant feline cardiomyopathic phenotype, often involves concentric left ventricular wall thickening rather than dilatational changes, and may not substantially enlarge the cardiac silhouette in early disease stages. VLAS has been shown to correlate with echocardiographic LA:Ao but with modest specificity, and multiple published studies have concluded that radiographic scores alone may be insufficient to substitute for echocardiography in B2 staging. Existing artificial intelligence tools for veterinary cardiac radiograph analysis may provide VHS measurements or binary triage findings, but may not predict echocardiography-derived metrics such as LA:Ao or provide the cross-modality predictions that could enable preliminary staging assessments at the point of care. Additionally, existing tools may require image upload through dedicated software or Picture Archiving and Communication System (PACS) integration, which may limit accessibility in some practice environments, and may output point estimates without spatial localization that would allow clinicians to validate automated assessments against the underlying image.

The systems and methods described with reference to FIGS. 18-23 may address these challenges through a two-stage prediction architecture that enables cross-modality prediction of echocardiography-derived metrics from radiographic images. In certain embodiments, a Stage A module executes a VHS and VLAS measurement pipeline to produce landmark coordinates, VHS values, and VLAS values from a preprocessed radiograph. A Stage B module may then receive as input one or more of the preprocessed radiograph image, the Stage A VHS and VLAS scalar values, the Stage A landmark coordinates, a heart silhouette segmentation mask, and intermediate feature maps from the Stage A model. The Stage B module may output a continuous predicted LA:Ao value with associated confidence, a binary B2 classification with associated probability, an ACVIM stage classification with class probabilities, a risk-of-progression score, and auxiliary predictions of echocardiography-derived metrics such as LVIDDN (left ventricular internal diameter in diastole normalized to body size). By combining radiographic scoring outputs with machine learning models trained on paired radiograph and echocardiography studies, the systems may provide preliminary staging assessments that were previously obtainable through echocardiography performed by a board-certified veterinary cardiologist.

The systems may further address veterinary cardiac assessment limitations (including feline cardiac assessment limitations) through species-specific modules configured to detect anatomical landmarks and predict disease status independent of conventional scoring systems. In certain embodiments, a veterinary disease prediction module may receive a preprocessed radiograph image and produce a disease status output indicating one or more of binary disease presence, categorical classification of cardiomyopathy type, or graded severity score. The veterinary disease prediction module may be trained to identify radiographic features predictive of cardiac disease without being constrained to VHS or VLAS as features or outputs, which may permit discovery of disease-correlated cardiac silhouette characteristics that existing scoring systems do not capture. This approach may address the limitation that VHS has moderate sensitivity for detecting hypertrophic cardiomyopathy in feline patients, where concentric left ventricular wall thickening may not substantially enlarge the cardiac silhouette in early disease stages.

The systems may also address workflow and interpretability limitations through a photograph-of-radiograph input pathway and an explainability module. In certain embodiments, the preprocessing pipeline may accept photographs of radiographs captured by a camera (e.g., a mobile phone camera) in addition to (or rather than) direct DICOM file uploads, which may enable use at general practice clinics without requiring dedicated software integration or PACS connectivity. The explainability module may generate spatial localization overlays on the radiograph indicating regions that contributed to model predictions, using one or more of Grad-CAM, integrated gradients, attention rollout for transformer architectures, or landmark and segmentation overlay highlighting the left atrial region. By providing spatial localization that clinicians may validate against the underlying image, the systems may support clinical adoption of cross-modality predictions while preserving veterinarian interpretive authority over clinical decision-making.

The systems and methods described with reference to FIGS. 18-23 may provide one or more of the following benefits. In certain embodiments, the two-stage prediction architecture may enable cross-modality prediction of echocardiography-derived metrics such as LA:Ao from radiographic images, which may provide preliminary staging assessments at the point of care without requiring access to specialized echocardiography equipment or board-certified veterinary cardiologists. By enabling LA:Ao and B2 classification predictions from radiographs that may be returned in seconds during a general practice visit, the systems may reduce diagnostic latency and accelerate identification of patients who may benefit from treatment. In some embodiments, the photograph-of-radiograph input pathway may lower barriers to adoption by enabling use at general practice clinics without requiring dedicated software integration or PACS connectivity. The veterinary disease prediction module may address limitations of conventional scoring systems by identifying radiographic features predictive of cardiac disease independent of VHS or VLAS, which may permit detection of conditions such as hypertrophic cardiomyopathy where concentric left ventricular wall thickening may not substantially enlarge the cardiac silhouette in early disease stages.

In certain embodiments, the explainability module may support clinical adoption by generating spatial localization overlays that indicate regions of the radiograph contributing to model predictions, which may allow clinicians to validate automated assessments against the underlying image. The systems may preserve veterinarian interpretive authority by outputting measurements and predictions with confidence values rather than generating treatment recommendations or referral instructions. In some embodiments, the systems may reduce the cost burden associated with cardiac staging by enabling screening and triage from radiographs, with echocardiography reserved for confirmation in appropriate cases. The flexible deployment options may enable use across a variety of practice environments and connectivity conditions. Additionally, the disclosed systems and methods applicable to feline patients may extend cardiac assessment capabilities to a population where existing radiographic scoring approaches may have limited sensitivity.

In certain embodiments, the systems and methods described herein may be applied to various clinical use cases beyond diagnostic triage at general practice visits. Alternative clinical use cases may include pre-anesthetic cardiac screening before surgical procedures to identify patients at elevated risk of anesthetic complications due to undiagnosed cardiac disease. The systems may be used for post-treatment monitoring to assess response to cardiac medications such as pimobendan, atenolol, or ACE inhibitors by tracking changes in VHS, VLAS, or predicted LA:Ao over time. In some embodiments, the systems may be used for insurance underwriting or actuarial risk scoring to assess cardiac disease risk in companion animals. The systems may also be applied to telemedicine and teleradiology triage, enabling remote assessment of radiographs submitted by general practitioners to specialists. In certain embodiments, the systems may be used for clinical trial enrollment screening to identify preclinical patients meeting study criteria for cardiac disease research. The systems may further be applied to population-level cardiac disease surveillance to monitor trends in cardiac disease prevalence across geographic regions or patient populations.

FIG. 18 illustrates a preprocessing process 1800 for radiograph images. In certain embodiments, one or more preprocessing steps may be applied to prepare radiograph images for downstream AI processing, including landmark detection, VHS and VLAS measurement, disease prediction, and cross-modality prediction of echocardiography-derived metrics.

The preprocessing process 1800 may begin at a step 1802 of receiving an input image. The input image may comprise one or more of a (i) photograph of a radiograph captured by a camera, such as a mobile phone camera, (also referred to as a photograph-of-radiograph or PoP input), (ii) a direct DICOM (Digital Imaging and Communications in Medicine) file upload, or (iii) other digital image formats. In some embodiments, the input image may comprise a right lateral thoracic radiograph, a left lateral thoracic radiograph, or both views.

At a step 1804, the preprocessing process 1800 may perform skew detection for correcting image orientation. Skew detection may identify angular deviation of the image from a reference orientation and may determine a correction angle to align the image with expected anatomical orientations.

At a step 1806, the preprocessing process 1800 may perform cropping to a region of interest for isolating relevant anatomy. Cropping to the region of interest may remove extraneous portions of the image and may isolate the thoracic region containing the cardiac silhouette and thoracic vertebrae. In some embodiments, the region of interest may be identified using one or more of edge detection, anatomical landmark detection, or machine learning-based region proposal.

At a step 1808, the preprocessing process 1800 may apply CLAHE (contrast-limited adaptive histogram equalization) for image enhancement. CLAHE may enhance local contrast within the radiograph image while limiting amplification of noise. In some embodiments, CLAHE may improve visibility of anatomical structures including the cardiac silhouette, vertebral bodies, carina, and left atrial border.

At a step 1810, the preprocessing process 1800 may perform perspective detection and correction. Step 1810 may apply to photograph-of-radiograph inputs where geometric distortions may be introduced during image capture. Perspective correction may identify and compensate for trapezoidal distortion, keystone effects, or other perspective artifacts that may result from capturing a photograph of a radiograph displayed on a viewing screen at an oblique angle. In some embodiments, step 1810 may be omitted for direct DICOM file inputs that do not exhibit perspective distortion.

At a step 1812, the preprocessing process 1800 may perform rotation correction for proper image alignment. Rotation correction may orient the radiograph image such that anatomical structures are aligned with expected orientations for downstream processing. In some embodiments, rotation correction may align the vertebral column along a reference axis.

At a step 1814, the preprocessing process 1800 may perform letterboxed resizing to a target resolution. Letterboxed resizing may scale the image while preserving the aspect ratio of the original image. Letterboxing may add padding to the shorter dimension to produce a standardized output size without distorting anatomical proportions. In some embodiments, the target resolution may be selected to balance computational efficiency with preservation of anatomical detail sufficient for landmark detection and measurement.

In certain embodiments, the preprocessing steps 1802 through 1814 may be performed sequentially as illustrated in FIG. 18. In other embodiments, one or more of the preprocessing steps may be omitted, reordered, or combined based on the characteristics of the input image. For example, perspective detection and correction at step 1810 may be omitted when the input image comprises a direct DICOM file upload rather than a photograph-of-radiograph input. The preprocessed radiograph image produced by the preprocessing process 1800 may be provided to downstream modules including a VHS and VLAS measurement pipeline, a disease prediction module, or a two-stage LA:Ao and B2 prediction system.

In certain embodiments, the systems and methods described herein may receive input images from imaging modalities other than conventional radiography. Alternative input modalities may include computed tomography (CT) imaging, which may provide cross-sectional views of the thorax and cardiac structures. The systems may also receive fluoroscopy images or video sequences capturing dynamic cardiac motion. In some embodiments, the systems may receive ultrasound stills as input, which may be processed using similar preprocessing and prediction pipelines adapted for ultrasound image characteristics. The systems may employ multimodal fusion combining inputs from two or more imaging modalities to improve prediction accuracy. In certain embodiments, the systems may support single-view operation using only a right lateral or left lateral thoracic radiograph, in addition to the dual-view operation using both right lateral and left lateral views.

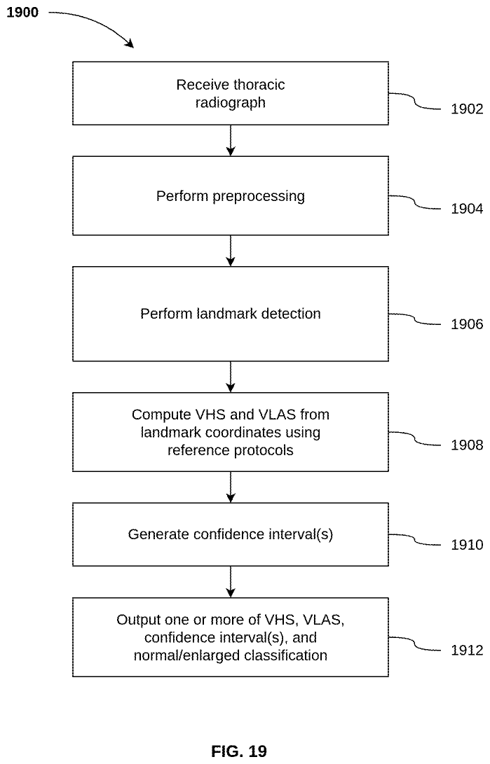

FIG. 19 illustrates a veterinary cardiac assessment process 1900. In certain embodiments, the veterinary cardiac assessment process 1900 may be configured to receive veterinary thoracic radiograph images and produce VHS and VLAS measurements using species-specific anatomical landmark detection and reference ranges.

The veterinary cardiac assessment process 1900 may begin at a step 1902 of receiving a veterinary thoracic radiograph (for example, and without limitation, a feline or canine thoracic radiograph). The veterinary thoracic radiograph may comprise one or more of a photograph of a radiograph (POP) captured by a mobile phone camera or a DICOM file uploaded directly from radiography equipment. In some embodiments, the veterinary thoracic radiograph may comprise a right lateral thoracic radiograph, a left lateral thoracic radiograph, or both views.

At a step 1904, the veterinary cardiac assessment process 1900 may perform preprocessing on the received veterinary thoracic radiograph. Preprocessing at step 1904 may include one or more of skew detection, cropping to a region of interest, CLAHE (contrast-limited adaptive histogram equalization), perspective correction for photograph-of-radiograph inputs, rotation correction, or letterboxed resizing to a target resolution. In some embodiments, the preprocessing operations at step 1904 may correspond to the preprocessing operations described with reference to FIG. 18.

At a step 1906, the veterinary cardiac assessment process 1900 may perform landmark detection. In certain embodiments, landmark detection at step 1906 may identify feline-specific anatomical reference points including one or more of thoracic vertebrae T4 onward, a cardiac silhouette long axis, a cardiac silhouette short axis, a carina, and a left atrial caudal border. In certain embodiments, the landmark detection may employ one or more of CNN-based landmark regression, keypoint detection networks, vision transformer architectures with regression heads, or a two-stage architecture comprising an anatomical region detector followed by a keypoint regressor. The landmark detection at step 1906 may account for anatomical differences between feline and canine thoracic structures.

At a step 1908, the veterinary cardiac assessment process 1900 may compute VHS and VLAS values from the landmark coordinates detected at step 1906. In certain embodiments, the VHS computation at step 1908 may use a feline VHS protocol with species-specific reference ranges of approximately 6.7 to 8.1 vertebrae. The veterinary cardiac assessment process 1900 may apply a cardiomegaly cutoff of VHS greater than 8.0 for cats. In some embodiments, the VLAS computation at step 1908 may use a feline-adapted VLAS protocol. The VHS and VLAS values may be computed analytically from the detected landmark coordinates.

At a step 1910, the veterinary cardiac assessment process 1900 may generate confidence intervals for the computed VHS and VLAS values. The confidence intervals may indicate a range of values within which the true VHS or VLAS measurement may fall with a specified probability. In some embodiments, the confidence intervals may be derived from one or more of model uncertainty estimates, ensemble predictions, or Monte Carlo dropout sampling.

At a step 1912, the veterinary cardiac assessment process 1900 may output results including one or more of a numeric VHS value, a numeric VLAS value, confidence intervals for the VHS and VLAS values, and a categorical normal or enlarged classification. In certain embodiments, the categorical classification at step 1912 may compare the computed VHS and VLAS values against feline reference ranges to determine whether the values indicate normal cardiac dimensions or cardiac enlargement. In certain embodiments, the output at step 1912 may be displayed on a user interface of a digital device running a mobile or online application.

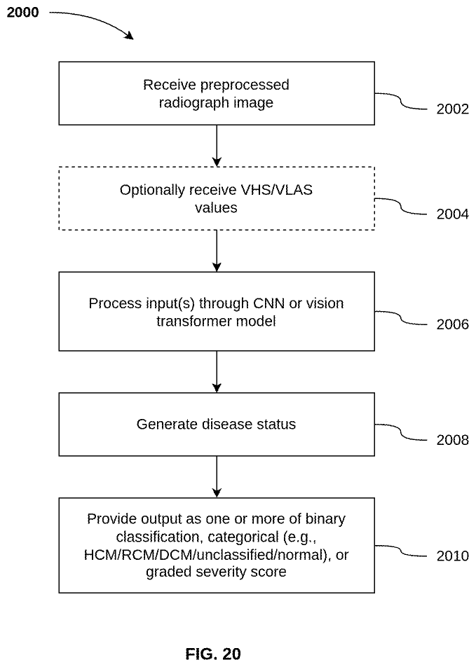

FIG. 20 illustrates a veterinary disease prediction process 2000. In certain embodiments, the veterinary disease prediction process 2000 may be configured to receive veterinary radiograph images (for example, and without limitation, feline or canine radiograph images) and produce disease status outputs that may be independent of conventional VHS and VLAS scoring systems.

The veterinary disease prediction process 2000 may begin at a step 2002 of receiving a preprocessed veterinary radiograph image. The preprocessed veterinary radiograph image may comprise a veterinary thoracic radiograph that has undergone one or more preprocessing operations including skew detection, cropping to a region of interest, CLAHE (contrast-limited adaptive histogram equalization), perspective correction for photograph-of-radiograph inputs, rotation correction, or letterboxed resizing. In some embodiments, the preprocessed veterinary radiograph image may correspond to the output of the preprocessing process 1800 described with reference to FIG. 18.

At a step 2004, the veterinary disease prediction process 2000 may optionally receive VHS and VLAS values as auxiliary features. The VHS and VLAS values at step 2004 may comprise outputs from the veterinary cardiac assessment process 1900 described with reference to FIG. 19. In certain embodiments, the VHS and VLAS values may be provided to a downstream model as supplemental input features alongside the preprocessed veterinary radiograph image. In other embodiments, step 2004 may be omitted, and the veterinary disease prediction process 2000 may operate on the preprocessed veterinary radiograph image without receiving VHS or VLAS values as auxiliary features. For example, though VHS and VLAS values may pose some benefit for feline patients, the predictive value of these measurements is lower with respect to felines than it is with respect to canines; for that reason, in certain example feline embodiments (but not all feline embodiments), an input score may be omitted.

At a step 2006, the veterinary disease prediction process 2000 may analyze the preprocessed veterinary radiograph image through a convolutional neural network (CNN) or vision transformer model trained on disease status labels. In certain embodiments, the CNN at step 2006 may comprise one or more of ResNet, EfficientNet, ConvNeXt, DenseNet, or Inception family architectures with classification or regression heads. In some embodiments, the vision transformer model at step 2006 may comprise one or more of ViT, Swin, or DeiT architectures with classification or regression heads. In certain embodiments, the model at step 2006 may employ multi-task heads predicting VHS, VLAS, and disease status jointly. Multi-task architectures may enable the model to learn shared representations across measurement and classification tasks, and may leverage cardiologist-annotated keypoints and VHS and VLAS values as auxiliary supervisory signals during training. In some embodiments, the model at step 2006 may employ segmentation-based feature extraction using one or more of U-Net, Attention U-Net, nnU-Net, Mask R-CNN, or SAM/MedSAM architectures followed by morphological feature analysis.

At a step 2008, the veterinary disease prediction process 2000 may generate a disease status output. The disease status output at step 2008 may indicate one or more of binary disease presence or absence, categorical disease classification, graded severity score, or any other suitable disease status output. In certain embodiments, the model may be trained to identify radiographic features predictive of cardiac disease without being constrained to VHS or VLAS as features or outputs, which may permit discovery of disease-correlated cardiac silhouette characteristics including shape, dimensions, internal contour, and regional density that conventional scoring systems may not capture.

At a step 2010, the veterinary disease prediction process 2000 may output classification results. In certain embodiments, the output at step 2010 may comprise a binary classification indicating disease present or disease absent. In some embodiments, the output at step 2010 may comprise a categorical disease classification including one or more of hypertrophic cardiomyopathy (HCM), restrictive cardiomyopathy (RCM), dilated cardiomyopathy (DCM), unclassified cardiomyopathy, or normal. In other embodiments, the output at step 2010 may comprise a graded severity score indicating a degree of disease progression. In certain embodiments, the output at step 2010 may include associated probability values or confidence scores for each classification category. The classification results at step 2010 may be displayed on a user interface of a digital device running a mobile or online application.

The veterinary disease prediction process 2000 may address limitations of conventional radiographic scoring systems for feline cardiac assessment. Hypertrophic cardiomyopathy, which represents a dominant feline cardiomyopathic phenotype, may involve concentric left ventricular wall thickening rather than dilatational changes, and may not substantially enlarge the cardiac silhouette in early disease stages. By training the model to identify radiographic features predictive of cardiac disease independent of VHS or VLAS, the veterinary disease prediction process 2000 may detect conditions where concentric left ventricular wall thickening may not produce cardiac silhouette enlargement detectable by conventional scoring methods.

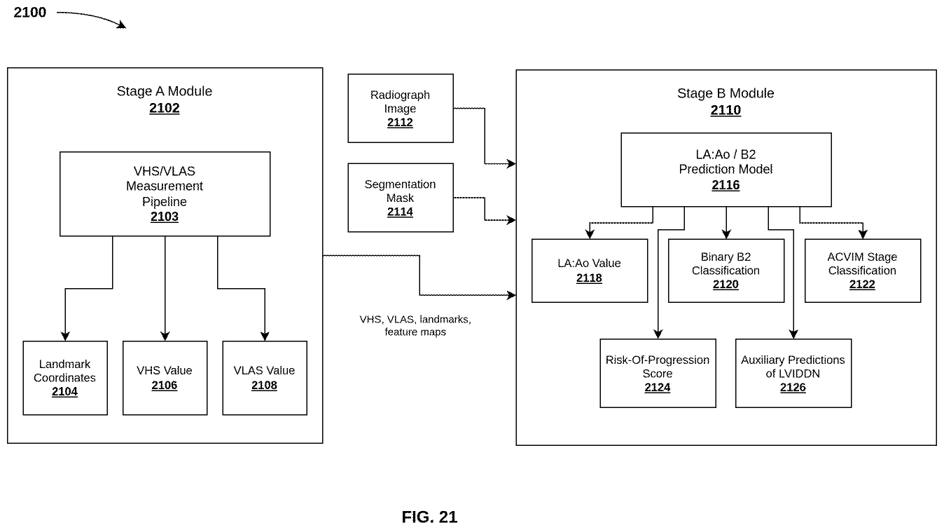

FIG. 21 illustrates a block diagram of a two-stage prediction system 2100 for LA:Ao and B2 prediction. In certain embodiments, the two-stage prediction system 2100 may be configured to receive preprocessed radiograph images and produce cross-modality predictions of echocardiography-derived metrics from radiographic inputs.

The two-stage prediction system 2100 may include a Stage A module 2102. The Stage A module 2102 may enclose a VHS/VLAS measurement pipeline 2103. In certain embodiments, the VHS/VLAS measurement pipeline 2103 may execute landmark detection and measurement operations on a preprocessed radiograph to produce downstream outputs. The VHS/VLAS measurement pipeline 2103 may produce landmark coordinates 2104, a VHS value 2106, and a VLAS value 2108. The landmark coordinates 2104 may comprise coordinates of anatomical reference points including one or more of the carina, the cardiac apex, the cardiac silhouette borders, the left atrial caudal border, and thoracic vertebrae. The VHS value 2106 may comprise a numeric vertebral heart score computed from the landmark coordinates 2104. The VLAS value 2108 may comprise a numeric vertebral left atrial score computed from the landmark coordinates 2104.

The two-stage prediction system 2100 may include a Stage B module 2110. The Stage B module 2110 may receive as input one or more of a radiograph image 2112, a heart silhouette segmentation mask 2114, and outputs from the Stage A module 2102. The radiograph image 2112 may comprise a thoracic radiograph that has undergone preprocessing operations including one or more of skew detection, cropping, contrast enhancement, perspective correction, rotation correction, and resizing. In certain embodiments, a radiograph image that has not been preprocessed may be provided in addition to or instead of a preprocessed radiograph image. The heart silhouette segmentation mask 2114 may comprise a binary or multi-class mask delineating the cardiac silhouette region within a preprocessed radiograph image. In certain embodiments, the Stage B module 2110 may receive the VHS value 2106, the VLAS value 2108, the landmark coordinates 2104, and intermediate feature maps from the Stage A module 2102 as additional inputs. Certain embodiments may omit either the Stage A module 2102 or the Stage B module 2110 altogether.

The Stage B module 2110 may include a left atrial enlargement prediction machine learning model 2116. The left atrial enlargement prediction machine learning model 2116 may process the inputs received by the Stage B module 2110 to produce prediction outputs. In certain embodiments, the left atrial enlargement prediction machine learning model 2116 may produce one or more of an LA:Ao value 2118, a binary B2 classification 2120, an ACVIM stage classification 2122, a risk-of-progression score 2124, and auxiliary predictions of LVIDDN 2126.

The LA:Ao value 2118 may comprise a continuous predicted left atrial-to-aortic ratio. In certain embodiments, the LA:Ao value 2118 may include an associated confidence interval indicating a range of values within which the true LA:Ao measurement may fall with a specified probability. The confidence interval may be derived from one or more of model uncertainty estimates, ensemble predictions, or Monte Carlo dropout sampling.

The binary B2 classification 2120 may comprise a prediction indicating whether a patient meets criteria for ACVIM Stage B2 classification. In certain embodiments, the binary B2 classification 2120 may include an associated probability value indicating the model confidence in the classification.

In certain embodiments, the binary B2 classification 2120 may be based on ACVIM 2019 consensus criteria for Stage B2 classification. Per the ACVIM 2019 consensus, a dog may be classified as ACVIM Stage B2 (asymptomatic preclinical myxomatous mitral valve disease with cardiac remodeling) when all of the following criteria are met: echocardiographic LA:Ao greater than or equal to 1.6, echocardiographic LVIDDN greater than or equal to 1.7, radiographic VHS greater than 10.5, and heart murmur intensity greater than or equal to 3/6. The two-stage prediction system 2100 may predict whether a patient meets the echocardiographic criteria (LA:Ao and LVIDDN) from radiographic inputs, enabling preliminary B2 screening at the point of care without requiring echocardiography.

The ACVIM stage classification 2122 may comprise a categorical classification indicating one or more of ACVIM Stage B1, Stage B2, Stage C, or Stage D. In certain embodiments, the ACVIM stage classification 2122 may include class probabilities for each stage category.

The risk-of-progression score 2124 may comprise a predicted probability of advancing to a worse ACVIM stage within a specified future time window. In some embodiments, the future time window may comprise 6 months or 12 months.

The auxiliary predictions of LVIDDN 2126 may comprise predicted values for left ventricular internal diameter in diastole normalized to body size (LVIDDN). In some embodiments, the auxiliary predictions of LVIDDN 2126 may include other echocardiography-derived metrics.

In certain embodiments, the two-stage prediction system 2100 may output a severity score for cardiac disease assessment. The severity score may comprise an ordinal scale, such as a 1-5 scale, or a continuous scale, such as a 0-100 scale. The severity score may indicate a degree of cardiac disease progression based on the radiographic features and Stage A outputs processed by the left atrial enlargement prediction machine learning model 2116.

In certain embodiments, the Stage B module 2110 may employ multi-input fusion networks combining image features extracted from the radiograph image 2112 and the heart silhouette segmentation mask 2114 with scalar and coordinate outputs from the Stage A module 2102. The multi-input fusion networks may concatenate, add, or otherwise combine feature representations from multiple input sources to produce a unified representation for prediction.

In some embodiments, the Stage B module 2110 may employ multi-task networks predicting the LA:Ao value 2118, the auxiliary predictions of LVIDDN 2126, the binary B2 classification 2120, and the risk-of-progression score 2124 jointly. Multi-task architectures may enable the left atrial enlargement prediction machine learning model 2116 to learn shared representations across measurement and classification tasks. In certain embodiments, multi-task training may leverage correlations between one or more of LA:Ao, LVIDDN, B2 stage, and risk-of-progression to improve prediction accuracy across outputs.

In certain embodiments, the system 2100 may employ various artificial intelligence algorithm architectures within the Stage A module 2102 and the Stage B module 2110. The Stage A module 2102 may employ one or more of CNN-based landmark regression, keypoint detection networks, vision transformer variants with regression heads, two-stage detector-regressor architectures, CNN classification or regression backbones, or segmentation-based feature extraction.

In some embodiments, the Stage A module 2102 may employ CNN-based landmark regression using one or more of ResNet, EfficientNet, or ConvNeXt backbones with regression heads. ResNet backbones may include ResNet-18, ResNet-34, ResNet-50, ResNet-101, or ResNet-152 variants. EfficientNet backbones may include EfficientNet-B0 through EfficientNet-B7 variants. ConvNeXt backbones may include ConvNeXt-Tiny, ConvNeXt-Small, ConvNeXt-Base, or ConvNeXt-Large variants. The regression heads may output coordinate values for anatomical landmarks detected within the preprocessed radiograph image. The example CNN-based landmark regression systems described above are given by way of example rather than limitation.

In certain embodiments, the Stage A module 2102 may employ keypoint detection networks including one or more of HRNet (High-Resolution Network), HigherHRNet, stacked hourglass networks, or DeepLabCut architectures. HRNet architectures may maintain high-resolution representations throughout the network to preserve spatial precision for landmark localization. HigherHRNet architectures may extend HRNet with bottom-up multi-scale feature aggregation. Stacked hourglass networks may employ repeated bottom-up and top-down processing with intermediate supervision to refine landmark predictions. DeepLabCut architectures may employ transfer learning from pose estimation models pretrained on large-scale datasets.

In some embodiments, the Stage A module 2102 may employ vision transformer variants including one or more of ViT (Vision Transformer), Swin Transformer, or DeiT (Data-efficient Image Transformer) architectures with regression heads. ViT architectures may divide the input image into patches and process the patches as a sequence using transformer encoder layers. Swin Transformer architectures may employ shifted window attention mechanisms to enable efficient processing of high-resolution images. DeiT architectures may employ knowledge distillation techniques to enable training on smaller datasets. The regression heads attached to the vision transformer variants may output landmark coordinates from the transformer output representations.

In certain embodiments, the Stage A module 2102 may employ a two-stage architecture comprising an anatomical region detector followed by a keypoint regressor. The anatomical region detector may identify bounding regions containing anatomical structures of interest within the preprocessed radiograph image. The keypoint regressor may then operate on the identified regions to predict precise landmark coordinates within each region. The two-stage architecture may enable coarse-to-fine localization of anatomical landmarks.

In some embodiments, the Stage A module 2102 may employ CNN classification or regression backbones including one or more of DenseNet or Inception family architectures. DenseNet architectures may include DenseNet-121, DenseNet-169, DenseNet-201, or DenseNet-264 variants. Inception family architectures may include Inception-v3, Inception-v4, or Inception-ResNet variants. The DenseNet and Inception backbones may extract feature representations from the preprocessed radiograph image for downstream landmark regression or measurement prediction. The example CNN classification or regression backbone systems described above are given by way of example rather than limitation.

In certain embodiments, the Stage A module 2102 may employ segmentation-based feature extraction using one or more of U-Net, Attention U-Net, nnU-Net, Mask R-CNN, or SAM/MedSAM (Segment Anything Model/Medical Segment Anything Model) architectures followed by morphological feature analysis. U-Net architectures may employ encoder-decoder structures with skip connections to produce segmentation masks of anatomical structures. Attention U-Net architectures may incorporate attention gates to focus on relevant anatomical regions. nnU-Net architectures may employ self-configuring segmentation pipelines that adapt to dataset characteristics. Mask R-CNN architectures may produce instance segmentation masks for individual anatomical structures. SAM and MedSAM architectures may employ foundation models pretrained on large-scale segmentation datasets. The morphological feature analysis may extract geometric measurements from the segmentation masks for VHS and VLAS computation. The example segmentation-based feature extraction systems described above are given by way of example rather than limitation.

In certain embodiments, the Stage B module 2110 may employ vision transformers operating on image input with scalar features concatenated to a classification token. The vision transformer may process the radiograph image 2112 or the heart silhouette segmentation mask 2114 as patch sequences. The scalar features may comprise one or more of the VHS value 2106, the VLAS value 2108, and coordinate values from the landmark coordinates 2104 produced by the Stage A module 2102. The scalar features may be concatenated to the classification token of the vision transformer prior to processing by transformer encoder layers. The left atrial enlargement prediction machine learning model 2116 may then produce prediction outputs from the combined image and scalar feature representations.

In certain embodiments, the system 2100 may output auxiliary metric predictions in addition to the auxiliary predictions of LVIDDN 2126. The auxiliary metric predictions may include one or more of a left ventricular (LV) mass index and an ejection fraction estimate. The LV mass index may comprise a predicted value for left ventricular mass normalized to body size. The ejection fraction estimate may comprise a predicted percentage value indicating the proportion of blood ejected from the left ventricle during each cardiac contraction. In some embodiments, the LV mass index and the ejection fraction estimate may be derived from radiographic features processed by the Stage B module 2110 in conjunction with outputs from the Stage A module 2102. The auxiliary metric predictions may include associated confidence intervals or probability values indicating model uncertainty.

In certain embodiments, the system 2100 may output longitudinal tracking outputs for monitoring cardiac status across multiple patient visits. The longitudinal tracking outputs may include one or more of a progression slope across multiple visits and a rate of cardiac silhouette change over time. The progression slope may comprise a computed trend value indicating the direction and magnitude of change in one or more cardiac measurements, such as VHS, VLAS, or predicted LA:Ao, across two or more radiograph studies obtained at different time points. The rate of cardiac silhouette change may comprise a computed value indicating the velocity of change in cardiac silhouette dimensions or morphology over a specified time interval. In some embodiments, the longitudinal tracking outputs may be computed by comparing current predictions with historical predictions stored in a database associated with a patient record. The longitudinal tracking outputs may enable veterinarians to assess disease trajectory and evaluate response to treatment over time.

In certain embodiments, the system 2100 may be configured to detect or predict one or more additional cardiac conditions beyond LA:Ao and B2 classification. The two-stage architecture described with reference to FIG. 21 may be extended to identify radiographic features associated with various cardiac and cardiopulmonary abnormalities.

In certain embodiments, the system 2100 may detect or predict left ventricular hypertrophy (LVH). The Stage B module 2110 may process the preprocessed radiograph image and outputs from the Stage A module to identify radiographic features associated with left ventricular wall thickening. The left atrial enlargement prediction machine learning model 2116 may include a classification head configured to output a binary LVH prediction or a graded LVH severity score. In some embodiments, the LVH prediction may be derived from cardiac silhouette morphology features, including shape characteristics and regional contour analysis, extracted by the Stage B module 2110.

In certain embodiments, the system 2100 may detect or predict pulmonary edema and congestive heart failure. The Stage B module 2110 may analyze lung field regions within the preprocessed radiograph image to identify radiographic patterns associated with pulmonary edema, including interstitial and alveolar infiltrates. The left atrial enlargement prediction machine learning model 2116 may include a classification head configured to output a binary congestive heart failure prediction or a categorical classification indicating severity of pulmonary edema. In some embodiments, the congestive heart failure prediction may incorporate both cardiac silhouette features from the Stage A module and lung field features extracted by the Stage B module 2110.

In certain embodiments, the system 2100 may detect or predict pleural effusion with cardiogenic versus non-cardiogenic distinction. The Stage B module 2110 may identify radiographic features associated with fluid accumulation in the pleural space. The left atrial enlargement prediction machine learning model 2116 may include a multi-class classification head configured to output one or more of a binary pleural effusion detection, a categorical classification distinguishing cardiogenic pleural effusion from non-cardiogenic pleural effusion, or a volumetric estimate of pleural fluid. In some embodiments, the cardiogenic versus non-cardiogenic distinction may be derived from a combination of cardiac silhouette features, including cardiac enlargement indicators from the Stage A module and pleural space features extracted by the Stage B module 2110.

In certain embodiments, the system 2100 may detect or predict pericardial effusion. The Stage B module 2110 may analyze the cardiac silhouette region within the preprocessed radiograph image to identify radiographic features associated with fluid accumulation in the pericardial space. The left atrial enlargement prediction machine learning model 2116 may include a classification head configured to output a binary pericardial effusion prediction or a graded severity score. In some embodiments, the pericardial effusion prediction may be derived from cardiac silhouette shape features, including globoid cardiac silhouette morphology, extracted by the Stage B module 2110.

In certain embodiments, the system 2100 may detect or predict vena caval distension. The Stage B module 2110 may analyze the caudal vena cava region within the preprocessed radiograph image to identify radiographic features associated with venous distension. The left atrial enlargement prediction machine learning model 2116 may include a regression head configured to output a predicted caudal vena cava diameter or a classification head configured to output a binary vena caval distension prediction. In some embodiments, the vena caval distension prediction may incorporate landmark coordinates from the Stage A module identifying the caudal vena cava borders.

In certain embodiments, the system 2100 may detect or predict right-sided cardiac enlargement. The Stage B module 2110 may analyze the cardiac silhouette region within the preprocessed radiograph image to identify radiographic features associated with right atrial or right ventricular enlargement. The left atrial enlargement prediction machine learning model 2116 may include a classification head configured to output a binary right-sided enlargement prediction or a categorical classification indicating the affected chamber. In some embodiments, the right-sided cardiac enlargement prediction may be derived from cardiac silhouette morphology features, including sternal contact length and right heart border contour, extracted by the Stage B module 2110.

In certain embodiments, the system 2100 may detect or predict pulmonary hypertension indicators. The Stage B module 2110 may analyze the pulmonary vasculature and cardiac silhouette regions within the preprocessed radiograph image to identify radiographic features associated with pulmonary hypertension. The left atrial enlargement prediction machine learning model 2116 may include a classification head configured to output a binary pulmonary hypertension prediction or a probability score indicating likelihood of pulmonary hypertension. In some embodiments, the pulmonary hypertension prediction may be derived from a combination of pulmonary artery enlargement features, right-sided cardiac enlargement features, and lung field features extracted by the Stage B module 2110.

In some embodiments, the system 2100 may output predictions for multiple cardiac conditions simultaneously using a multi-task architecture within the Stage B module 2110. The left atrial enlargement prediction machine learning model 2116 may include multiple classification and regression heads configured to produce concurrent predictions for two or more of left ventricular hypertrophy, pulmonary edema, congestive heart failure, pleural effusion, pericardial effusion, vena caval distension, right-sided cardiac enlargement, and pulmonary hypertension indicators. Multi-task training may enable the left atrial enlargement prediction machine learning model 2116 to learn shared feature representations across related cardiac conditions.

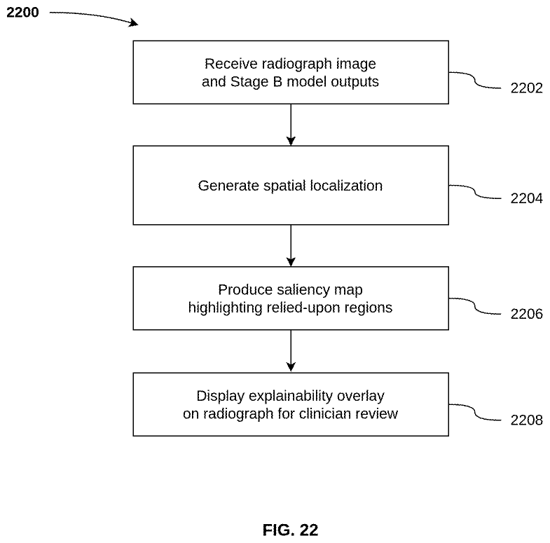

FIG. 22 illustrates an explainability process 2200 for cross-modality prediction. In certain embodiments, the explainability process 2200 may provide spatial localization information indicating regions of a radiograph image that contributed to model predictions, which may allow clinicians to validate automated assessments against the underlying image.

The explainability process 2200 may begin at a step 2202 of receiving a radiograph image and Stage B model outputs. The radiograph image at step 2202 may comprise a preprocessed radiograph image that has undergone one or more preprocessing operations including skew detection, cropping, contrast enhancement, perspective correction, rotation correction, and resizing. The Stage B model outputs at step 2202 may comprise one or more of an LA:Ao value, a binary B2 classification, an ACVIM stage classification, a risk-of-progression score, and auxiliary predictions of echocardiography-derived metrics such as LVIDDN.

At a step 2204, the explainability process 2200 may generate spatial localization. In certain embodiments, the spatial localization at step 2204 may employ one or more of Grad-CAM (Gradient-weighted Class Activation Mapping), Grad-CAM++, integrated gradients, or attention rollout for transformer architectures. Grad-CAM and Grad-CAM++ may compute gradient-based activation maps that highlight regions of the radiograph image that contributed to a particular model output. Integrated gradients may compute attribution scores by integrating gradients along a path from a baseline input to the actual input image. Attention rollout may aggregate attention weights across transformer layers to identify image regions that received attention during model processing. In some embodiments, the spatial localization at step 2204 may employ landmark or segmentation overlay highlighting the left atrial (LA) region.

At a step 2206, the explainability process 2200 may produce a saliency map highlighting relied-upon regions. The saliency map at step 2206 may comprise a visual representation indicating the relative contribution of different image regions to the model predictions. In certain embodiments, the saliency map may employ color gradients or intensity variations to indicate regions of higher or lower contribution to the model output. The saliency map may overlay the original radiograph image to provide spatial context for the highlighted regions.

At a step 2208, the explainability process 2200 may display the explainability overlay on a radiograph for clinician review. The explainability overlay at step 2208 may be presented on a user interface of a digital device running a mobile or online application. In certain embodiments, the explainability overlay may enable veterinarians to evaluate the plausibility of automated assessments by comparing the highlighted regions against the underlying radiograph image. The display at step 2208 may present the explainability overlay alongside the predicted values, confidence intervals, and classification outputs produced by the Stage B module. In some embodiments, the user interface may allow clinicians to toggle the explainability overlay on or off, adjust overlay transparency, or select different explainability visualization methods for display.

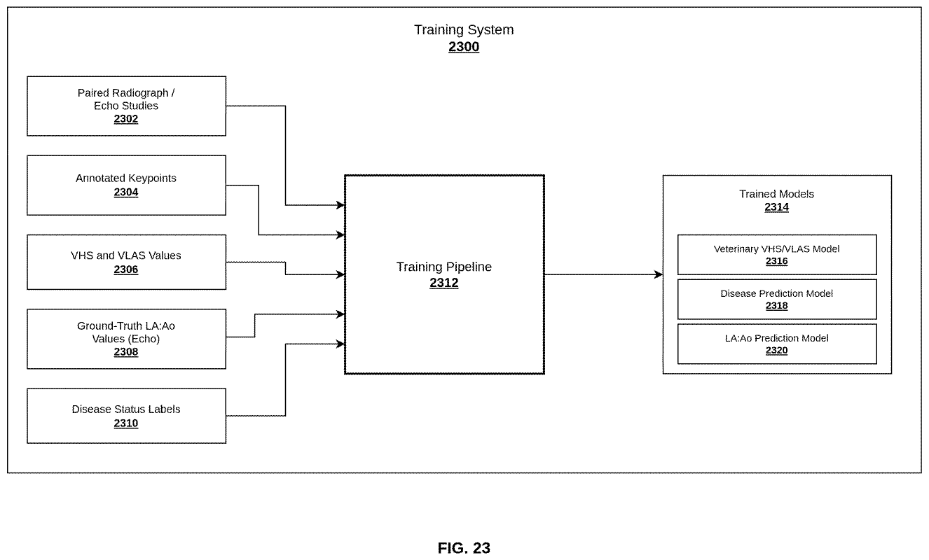

FIG. 23 illustrates a block diagram of a training system 2300 for the AI models. In certain embodiments, the training system 2300 may be configured to receive labeled data inputs and produce trained models for cardiac assessment and cross-modality prediction.

The training system 2300 may receive multiple labeled data inputs arranged on a left side of the training system 2300. The labeled data inputs may include paired radiograph and echocardiography studies 2302. The paired studies 2302 may comprise thoracic radiograph images obtained from the same patient within a temporal proximity to echocardiography examinations. In certain embodiments, the paired studies 2302 may include right lateral thoracic radiographs, left lateral thoracic radiographs, or both views, along with corresponding echocardiography data obtained from the same patient.

The labeled data inputs may include cardiologist-annotated keypoints 2304. The cardiologist-annotated keypoints 2304 may comprise coordinate annotations identifying anatomical reference points within radiograph images. In certain embodiments, the cardiologist-annotated keypoints 2304 may include annotations for one or more of the carina, the cardiac apex, the cardiac silhouette borders, the left atrial caudal border, and thoracic vertebrae. The cardiologist-annotated keypoints 2304 may be provided by board-certified veterinary cardiologists or other trained annotators.

The labeled data inputs may include VHS and VLAS values 2306. The VHS and VLAS values 2306 may comprise cardiologist-derived vertebral heart score and vertebral left atrial score measurements computed from the radiograph images. In certain embodiments, the VHS and VLAS values 2306 may serve as supervisory signals for training landmark detection and measurement models.

The labeled data inputs may include ground-truth LA:Ao values 2308. The ground-truth LA:Ao values 2308 may comprise left atrial-to-aortic ratio measurements obtained from echocardiography examinations. In certain embodiments, the ground-truth LA:Ao values 2308 may be measured on a right parasternal short-axis view at the heart base in early diastole by a board-certified veterinary cardiologist. The ground-truth LA:Ao values 2308 may provide supervisory signals for training cross-modality prediction models.

The labeled data inputs may include disease status labels 2310. The disease status labels 2310 may comprise cardiologist-assigned classifications indicating cardiac disease presence, type, or severity. In certain embodiments, the disease status labels 2310 may include one or more of binary disease presence or absence, categorical disease classification (such as hypertrophic cardiomyopathy, restrictive cardiomyopathy, dilated cardiomyopathy, unclassified cardiomyopathy, or normal), ACVIM stage classification, or graded severity scores. The disease status labels 2310 may be confirmed by echocardiography examination.

The training system 2300 may include a training pipeline 2312. The training pipeline 2312 may receive the labeled data inputs including the paired studies 2302, the cardiologist-annotated keypoints 2304, the VHS and VLAS values 2306, the ground-truth LA:Ao values 2308, and the disease status labels 2310. In certain embodiments, the training pipeline 2312 may process the labeled data inputs to train one or more machine learning models for cardiac assessment and prediction tasks.

In certain embodiments, the training pipeline 2312 may record a time delta between paired radiograph and echocardiography studies within the paired studies 2302. The time delta may indicate the temporal interval between acquisition of the radiograph image and performance of the echocardiography examination for a given patient. In some embodiments, the training pipeline 2312 may exclude pairs with time deltas exceeding a threshold interval, where the threshold interval may represent a clinically meaningful duration during which cardiac status may change. In other embodiments, the training pipeline 2312 may weight pairs based on the time delta, with pairs having shorter time deltas receiving higher weights during training and pairs having longer time deltas receiving lower weights. The exclusion or weighting of pairs based on time delta may reduce the influence of training examples where the radiograph and echocardiography measurements may not reflect the same cardiac state.

In certain embodiments, the training pipeline 2312 may employ k-fold cross-validation with a held-out test set for model validation. K-fold cross-validation may partition the training data into k subsets, with the training pipeline 2312 training k separate models, each using k−1 subsets for training and one subset for validation. The held-out test set may comprise a portion of the labeled data that is not used during training or cross-validation, and may be reserved for final evaluation of model performance. In some embodiments, the training pipeline 2312 may use stratified sampling to ensure that each fold and the held-out test set contain representative distributions of disease categories, species, breeds, or other relevant characteristics.

In certain embodiments, the training pipeline 2312 may compare model outputs against board-certified cardiologist measurements or echocardiography-confirmed diagnosis as a gold standard. For VHS and VLAS measurement models, the gold standard may comprise cardiologist-derived VHS and VLAS values computed from the same radiograph images. For LA:Ao prediction models, the gold standard may comprise the ground-truth LA:Ao values 2308 obtained from echocardiography. For disease prediction models, the gold standard may comprise echocardiography-confirmed diagnosis provided by a board-certified veterinary cardiologist. Comparison against the gold standard may enable computation of performance metrics including accuracy, sensitivity, specificity, area under the receiver operating characteristic curve (AUC-ROC), mean absolute error, or other metrics appropriate to the prediction task.

The training pipeline 2312 may produce trained models 2314. The trained models 2314 may be stored in a container on a right side of the training system 2300. In certain embodiments, the trained models 2314 may include a veterinary VHS/VLAS model 2316, a disease prediction model 2318, and an LA:Ao prediction model 2320.

In certain embodiments, LA:Ao prediction model 2320 may determine only a LA:Ao ratio. In certain other embodiments, LA:Ao prediction model 2320 may be a left atrial enlargement prediction machine learning model configured to provide a prediction as to whether the patient has left atrial enlargement.

The veterinary VHS/VLAS model 2316 may comprise a trained model configured to detect feline-specific anatomical landmarks and compute VHS and VLAS measurements from feline thoracic radiograph images. In certain embodiments, the veterinary VHS/VLAS model 2316 may be trained using the cardiologist-annotated keypoints 2304 and the VHS and VLAS values 2306 obtained from feline patients.

The disease prediction model 2318 may comprise a trained model configured to predict cardiac disease status from radiograph images. In certain embodiments, the disease prediction model 2318 may be trained using the disease status labels 2310 as supervisory signals. The disease prediction model 2318 may output one or more of binary disease classification, categorical disease classification, or graded severity scores.

The LA:Ao prediction model 2320 may comprise a trained model configured to predict left atrial-to-aortic ratio values from radiograph images. In certain embodiments, the LA:Ao prediction model 2320 may be trained using the ground-truth LA:Ao values 2308 obtained from echocardiography as supervisory signals. The LA:Ao prediction model 2320 may correspond to the left atrial enlargement prediction machine learning model 2116 described with reference to FIG. 21.

In certain embodiments, the trained models 2314 may be deployed via one or more deployment pathways for use in clinical settings. The deployment pathways may include cloud-based platforms, on-device inference on mobile phones or tablets, embedded integration with radiography equipment, PACS plugin integration, or hybrid edge and cloud architectures.

In certain embodiments, the trained models 2314 may include breed-specific sub-models and age-stratified sub-models. Breed-specific sub-models may be trained on data from specific breeds that have elevated prevalence of cardiac disease or breed-specific cardiac morphology, including but not limited to Cavalier King Charles Spaniel, Doberman, Maine Coon, Boxer, and Sphynx. The breed-specific sub-models may account for breed-specific variations in normal cardiac dimensions, cardiac silhouette shape, and disease prevalence. Age-stratified sub-models may be trained separately for pediatric, adult, and geriatric patient populations to account for age-related changes in cardiac morphology and disease presentation. In some embodiments, the system may automatically select the appropriate breed-specific or age-stratified sub-model based on patient metadata provided by the user.

In certain embodiments, the training pipeline 2312 may employ one or more training paradigms to produce the trained models 2314. The training paradigms may include one or more of transfer learning, self-supervised pretraining, federated learning, active learning, synthetic data augmentation, contrastive learning, or domain adaptation.

In certain embodiments, the training pipeline 2312 may employ transfer learning from radiology foundation models. Radiology foundation models may comprise neural network models pretrained on large-scale medical imaging datasets. For example, and without limitation, the radiology foundation models may include one or more of RadImageNet, MedSAM (Medical Segment Anything Model), or BiomedCLIP. RadImageNet may comprise a model pretrained on a large corpus of radiological images spanning multiple imaging modalities and anatomical regions. MedSAM may comprise a segmentation foundation model adapted for medical imaging applications. BiomedCLIP may comprise a vision-language model pretrained on biomedical image-text pairs. Transfer learning from radiology foundation models may initialize model weights with representations learned from large-scale medical imaging data, which may reduce the amount of labeled veterinary radiograph data required for training and may improve model generalization.

In certain embodiments, the training pipeline 2312 may employ transfer learning from ImageNet pretrained backbones. ImageNet pretraining may initialize model weights with representations learned from a large-scale natural image classification dataset comprising millions of labeled images across thousands of object categories. Transfer learning from ImageNet pretrained backbones may be used independently or in combination with subsequent fine-tuning on radiology-specific datasets or veterinary-specific datasets. In some embodiments, ImageNet pretraining may be followed by intermediate fine-tuning on radiology foundation models such as RadImageNet before final fine-tuning on the veterinary cardiac assessment task. ImageNet pretraining may provide a useful initialization for models when veterinary-specific training data is limited.

In certain embodiments, the training pipeline 2312 may employ self-supervised pretraining on unlabeled radiographs. Self-supervised pretraining may train a model to learn visual representations from unlabeled radiograph images without requiring cardiologist annotations. In some embodiments, the self-supervised pretraining may employ one or more of DINO (self-DIstillation with NO labels), MAE (Masked Autoencoder), SimCLR (Simple Contrastive Learning of Representations), or BYOL (Bootstrap Your Own Latent). DINO may employ self-distillation between student and teacher networks to learn visual representations. MAE may train a model to reconstruct masked portions of input images. SimCLR may employ contrastive learning between augmented views of the same image. BYOL may employ a momentum-based self-supervised learning approach without negative pairs. Self-supervised pretraining on unlabeled radiographs may enable the training pipeline 2312 to leverage large collections of veterinary radiograph images that lack cardiologist annotations.

In certain embodiments, the training pipeline 2312 may employ federated learning across multiple clinic partners. Federated learning may enable training of machine learning models across distributed datasets held by multiple veterinary clinics without transferring raw radiograph images or patient data between clinics. In federated learning, each participating clinic may train a local model on local data and transmit model updates, such as gradient updates or model weight updates, to a central server. The central server may aggregate the model updates from multiple clinics to produce a global model. Federated learning may enable privacy-preserving model training by keeping radiograph images and patient data at the originating clinic while still enabling collaborative model improvement across multiple clinic partners.

In certain embodiments, the training pipeline 2312 may employ active learning. Active learning may enable the training pipeline 2312 to select informative unlabeled cases for cardiologist labeling. In active learning, a model may evaluate unlabeled radiograph images and identify cases where the model exhibits high uncertainty or where additional labels may provide the greatest improvement in model performance. The identified cases may be presented to a board-certified veterinary cardiologist for annotation. By selecting informative cases for labeling, active learning may reduce the total number of cardiologist annotations required to achieve a target level of model performance.

In certain embodiments, the training pipeline 2312 may employ synthetic data augmentation. Synthetic data augmentation may generate synthetic radiograph images to supplement the labeled training data. In some embodiments, the synthetic radiograph images may be generated using one or more of generative adversarial networks (GANs) or diffusion models. GAN-generated radiographs may be produced by a generator network trained to produce realistic radiograph images that a discriminator network cannot distinguish from real radiograph images. Diffusion-generated radiographs may be produced by a diffusion model trained to iteratively denoise samples from a noise distribution to produce realistic radiograph images. Synthetic data augmentation may balance rare disease classes by generating additional synthetic examples of underrepresented disease categories, which may address class imbalance in the training data.

In certain embodiments, the training pipeline 2312 may employ contrastive learning between paired radiograph and echocardiogram embeddings. Contrastive learning may train a model to learn a cross-modal representation that aligns radiograph image embeddings with corresponding echocardiogram embeddings from the same patient. In contrastive learning, the model may be trained to maximize similarity between embeddings of paired radiograph and echocardiogram studies while minimizing similarity between embeddings of unpaired studies. The cross-modal representation learned through contrastive learning may enable the model to extract features from radiograph images that are predictive of echocardiography-derived metrics.

In certain embodiments, the training pipeline 2312 may employ domain adaptation. Domain adaptation may address variations across clinics, radiography equipment, and radiograph processing pipelines that may cause distribution shifts between training data and deployment data. In some embodiments, domain adaptation may employ one or more of adversarial domain adaptation, domain randomization, or feature alignment techniques. Adversarial domain adaptation may train a model to produce feature representations that a domain discriminator cannot distinguish between source and target domains. Domain randomization may apply random variations to training images to simulate the range of variations encountered across different clinics and equipment. Feature alignment techniques may align feature distributions between source and target domains to reduce the impact of domain shift on model performance. Domain adaptation may enable the trained models 2314 to generalize across clinic, equipment, and radiograph processing variations encountered in clinical deployment.

FIGS. 1-23 are provided by way of example rather than limitation. Elements may bee added, removed, rearranged, and/or modified without departing from the scope of the present disclosure.

In certain embodiments, a radiograph image corresponding to an animal may be received. The radiograph image may comprise one or more of a right lateral thoracic radiograph, a left lateral thoracic radiograph, or both views. The animal may be any suitable animal (for example, and without limitation, a dog, a cat, a rabbit, a ferret, or a horse). The radiograph image may be received in any suitable format, including but not limited to one or more of DICOM format, JPEG format, PNG format, TIFF format, or BMP format.

In certain embodiments, generating, using a left atrial enlargement prediction machine learning model, a left atrial enlargement prediction based on the radiograph image may comprise processing the inputs through a Stage B module 2110 as described with reference to FIG. 21. The Stage B module 2110 may receive as input one or more of the radiograph image 2112, a heart silhouette segmentation mask 2114, the VHS value 2106, the VLAS value 2108, the landmark coordinates 2104, and intermediate feature maps from the Stage A module 2102. The left atrial enlargement prediction machine learning model may comprise a left atrial enlargement prediction machine learning model 2116 that processes the inputs to produce prediction outputs.

In certain embodiments, the left atrial enlargement prediction may comprise a predicted left atrial-to-aortic ratio (LA:Ao) value. The LA:Ao value 2118 may comprise a continuous predicted left atrial-to-aortic ratio with an associated confidence interval. The confidence interval may indicate a range of values within which the true LA:Ao measurement may fall with a specified probability. In some embodiments, the confidence interval may be derived from one or more of model uncertainty estimates, ensemble predictions, or Monte Carlo dropout sampling.

In certain embodiments, the left atrial enlargement prediction may comprise a binary B2 classification indicating whether the animal meets criteria for ACVIM Stage B2 classification. The binary B2 classification 2120 may include an associated probability value indicating the model confidence in the classification. ACVIM Stage B2 may represent asymptomatic preclinical myxomatous mitral valve disease with cardiac remodeling.

In certain embodiments, the left atrial enlargement prediction may comprise an ACVIM stage classification indicating one or more of Stage B1, Stage B2, Stage C, or Stage D. The ACVIM stage classification 2122 may include class probabilities for each stage category. In some embodiments, the ACVIM stage classification may further include Stage A classification.

In certain embodiments, generating a risk-of-progression score indicating a predicted probability of the animal advancing to a worse ACVIM stage within a future time window may comprise outputting a risk-of-progression score 2124 from the left atrial enlargement prediction machine learning model 2116. The future time window may comprise any suitable time window (for example, and without limitation, 6 months or 12 months). The risk-of-progression score may enable veterinarians to assess disease trajectory and identify patients who may benefit from earlier intervention.

In certain embodiments, preprocessing the radiograph image prior to generating the left atrial enlargement prediction may comprise performing one or more preprocessing operations as described with reference to FIG. 18. The preprocessing process 1800 may include any one or more of receiving an input image at step 1802, performing skew detection at step 1804 for correcting image orientation, cropping to a region of interest at step 1806 for isolating relevant anatomy, applying CLAHE (contrast-limited adaptive histogram equalization) at step 1808 for image enhancement, performing perspective detection and correction at step 1810 for photograph-of-radiograph inputs, performing rotation correction at step 1812 for proper image alignment, and performing letterboxed resizing at step 1814 to a target resolution.

In certain embodiments, receiving the radiograph image may comprise receiving a photograph of a radiograph captured by a camera (for example, and without limitation, a camera of a mobile device). The photograph of a radiograph may be referred to as a photograph-of-radiograph or PoP input. If a mobile device is used to capture the POP input, the mobile device may comprise one or more of a smartphone, a tablet, or other portable computing device. In some embodiments, the preprocessing process 1800 may perform perspective detection and correction at step 1810 to compensate for trapezoidal distortion, keystone effects, or other perspective artifacts that may result from capturing a photograph of a radiograph displayed on a viewing screen at an oblique angle.

In certain embodiments, generating a spatial localization overlay indicating one or more regions of the radiograph image that contributed to the left atrial enlargement prediction may comprise executing an explainability process 2200 as described with reference to FIG. 22. The explainability process 2200 may generate spatial localization at step 2204 using one or more of Grad-CAM (Gradient-weighted Class Activation Mapping), Grad-CAM++, integrated gradients, or attention rollout for transformer architectures. The explainability process 2200 may produce a saliency map at step 2206 highlighting relied-upon regions and may overlay landmark and segmentation highlighting on the left atrial region at step 2208.

In certain embodiments, displaying the spatial localization overlay on the radiograph image may comprise presenting the explainability overlay on a user interface of a digital device running a mobile or online application at step 2208. The explainability overlay may enable veterinarians to evaluate the plausibility of automated assessments by comparing the highlighted regions against the underlying radiograph image. In some embodiments, the user interface may allow clinicians to toggle the explainability overlay on or off, adjust overlay transparency, or select different explainability visualization methods for display.

In certain embodiments, generating a confidence value associated with the left atrial enlargement prediction may comprise deriving confidence estimates from one or more of model uncertainty estimates, ensemble predictions, Monte Carlo dropout sampling, Bayesian neural networks that output posterior distributions over predictions, deep ensembles comprising multiple independently trained models whose predictions are aggregated, test-time augmentation in which multiple augmented versions of an input image are processed and predictions are aggregated, heteroscedastic regression models that predict both a mean value and an associated variance, or conformal prediction methods that produce prediction intervals with coverage guarantees.

The left atrial enlargement prediction machine learning model may comprise any suitable machine learning model. For example, and without limitation, the left atrial enlargement prediction machine learning model may comprise one or more of CNN regression heads operating on image and scalar feature input, vision transformers operating on image input with scalar features concatenated to a classification token, multi-input fusion networks combining image features and Stage A outputs, or multi-task networks predicting LA:Ao, LVIDDN, B2 stage, and risk-of-progression jointly. The CNN regression heads may employ one or more of ResNet, EfficientNet, ConvNeXt, DenseNet, or Inception family architectures. The vision transformers may employ one or more of ViT, Swin, or DeiT architectures.

In certain embodiments, the left atrial enlargement prediction may further comprise auxiliary predictions of echocardiography-derived metrics. The auxiliary predictions may include one or more of LVIDDN (left ventricular internal diameter in diastole normalized to body size) 2126, a left ventricular mass index, or an ejection fraction estimate. In some embodiments, the auxiliary predictions may include associated confidence intervals or probability values indicating model uncertainty.

In certain embodiments, the systems and methods may generate longitudinal tracking outputs for monitoring cardiac status across multiple patient visits. The longitudinal tracking outputs may include one or more of a progression slope across multiple visits or a rate of cardiac silhouette change over time. The progression slope may comprise a computed trend value indicating the direction and magnitude of change in one or more cardiac measurements across two or more radiograph studies obtained at different time points.

In certain embodiments, the systems and methods may employ multimodal fusion combining radiograph data with one or more additional data modalities. The additional data modalities may include one or more of electrocardiogram data, auscultation or phonocardiogram data, photoplethysmography data, NT-proBNP biomarker values, clinical metadata including signalment and body condition information, or owner-captured smartphone video of the patient. The multimodal fusion architectures may include one or more of early fusion, late fusion, intermediate fusion, or attention-based fusion mechanisms.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as outlined in the description. Although the present embodiments are shown above, they are not limited to just these embodiments but are amenable to various changes and modifications without departing from the spirit thereof.

1. A computer-implemented method for cardiac assessment in veterinary patients, comprising:
receiving a radiograph image corresponding to an animal;
preprocessing the radiograph image to generate a preprocessed radiograph image;
based on the preprocessed radiograph image, generating, using a left atrial enlargement prediction machine learning model, a left atrial enlargement prediction; and
providing access to the left atrial enlargement prediction.

2. The computer-implemented method of claim 1, wherein the left atrial enlargement prediction comprises one or more of:
a predicted left atrial-to-aortic ratio (LA:Ao) value;
a binary B2 classification indicating whether the animal meets criteria for ACVIM Stage B2 classification; or
an ACVIM stage classification indicating one or more of Stage B1, Stage B2, Stage C, or Stage D.

3. The computer-implemented method of claim 2, further comprising generating a risk-of-progression score indicating a predicted probability of the animal advancing to a worse ACVIM stage within a future time window.

4. The computer-implemented method of claim 1, wherein preprocessing the radiograph image comprises one or more of skew detection, cropping to a region of interest, contrast-limited adaptive histogram equalization (CLAHE), perspective correction, rotation correction, or letterboxed resizing, and wherein the animal is a feline or a canine.

5. The computer-implemented method of claim 1, wherein receiving the radiograph image comprises receiving a photograph of a radiograph captured by a camera of a mobile device.

6. The computer-implemented method of claim 1, further comprising:
generating a spatial localization overlay indicating one or more regions of the radiograph image or the preprocessed radiograph image that contributed to the left atrial enlargement prediction; and
displaying the spatial localization overlay on the radiograph image or the preprocessed radiograph image.

7. The computer-implemented method of claim 1, further comprising:
generating a confidence value associated with the left atrial enlargement prediction; and
providing access to the confidence value.

8. A system for cardiac assessment in veterinary patients, comprising:
at least one processor; and
memory storing instructions executable by the at least one processor for causing the system to perform operations comprising:
receiving a radiograph image corresponding to an animal;
preprocessing the radiograph image to generate a preprocessed radiograph image;
based on the preprocessed radiograph image, generating, using a left atrial enlargement prediction machine learning model, a left atrial enlargement prediction; and
providing access to the left atrial enlargement prediction.

9. The system of claim 8, wherein the left atrial enlargement prediction comprises one or more of:
a predicted left atrial-to-aortic ratio (LA:Ao) value;
a binary B2 classification indicating whether the animal meets criteria for ACVIM Stage B2 classification; or
an ACVIM stage classification indicating one or more of Stage B1, Stage B2, Stage C, or Stage D.

10. The system of claim 9, wherein the operations further comprise generating a risk-of-progression score indicating a predicted probability of the animal advancing to a worse ACVIM stage within a future time window.

11. The system of claim 8, wherein preprocessing the radiograph image comprises one or more of skew detection, cropping to a region of interest, contrast-limited adaptive histogram equalization (CLAHE), perspective correction, rotation correction, or letterboxed resizing, and wherein the animal is a feline or a canine.

12. The system of claim 8, wherein receiving the radiograph image comprises receiving a photograph of a radiograph captured by a camera of a mobile device.

13. The system of claim 8, wherein the operations further comprise:
generating a spatial localization overlay indicating one or more regions of the radiograph image or the preprocessed radiograph image that contributed to the left atrial enlargement prediction; and
displaying the spatial localization overlay on the radiograph image or the preprocessed radiograph image.

14. The system of claim 8, wherein the operations further comprise:
generating a confidence value associated with the left atrial enlargement prediction; and
providing access to the confidence value.

15. A non-transitory computer-readable medium having stored thereon program instructions that are executable by at least one processor for causing the at least one processor to perform operations comprising:
receiving a radiograph image corresponding to an animal;
preprocessing the radiograph image to generate a preprocessed radiograph image;
based on the preprocessed radiograph image, generating, using a left atrial enlargement prediction machine learning model, a left atrial enlargement prediction; and
providing access to the left atrial enlargement prediction.

16. The non-transitory computer-readable medium of claim 15, wherein the left atrial enlargement prediction comprises one or more of:
a predicted left atrial-to-aortic ratio (LA:Ao) value;
a binary B2 classification indicating whether the animal meets criteria for ACVIM Stage B2 classification; or
an ACVIM stage classification indicating one or more of Stage B1, Stage B2, Stage C, or Stage D.

17. The non-transitory computer-readable medium of claim 16, wherein the operations further comprise generating a risk-of-progression score indicating a predicted probability of the animal advancing to a worse ACVIM stage within a future time window.

18. The non-transitory computer-readable medium of claim 15, wherein preprocessing the radiograph image comprises one or more of skew detection, cropping to a region of interest, contrast-limited adaptive histogram equalization (CLAHE), perspective correction, rotation correction, or letterboxed resizing, and wherein the animal is a feline or a canine.

19. The non-transitory computer-readable medium of claim 15, wherein receiving the radiograph image comprises receiving a photograph of a radiograph captured by a camera of a mobile device.

20. The non-transitory computer-readable medium of claim 15, wherein the operations further comprise:
generating a confidence value associated with the left atrial enlargement prediction; and
providing access to the confidence value.
